(19) United States
(12) Patent Application Publication
(10) Pub. No.: US 2025/0011366 A1
(43) Pub. Date: Jan. 9, 2025

(54) Prodrugs of Antibiotic Teixobactin

(71) Applicant: The Regents of the University of California, Oakland, CA (US)

(72) Inventors: Chelsea R. Jones, Irvine, CA (US); James S. Nowick, Irvine, CA (US)

(73) Assignee: The Regents of the University of California, Oakland, CA (US)

(21) Appl. No.: 18/690,563

(22) PCT Filed: Sep. 8, 2022
(86) PCT No.: PCT/US2022/076130

§ 371 Date: Mar. 8, 2024

Related U.S. Application Data
(60) Provisional application No. 63/364,690, filed on May 13, 2022.
(60) Provisional application No. 63/241,920, filed on Sep. 8, 2021.

(51) Int. Cl. C07K 7/56; A61K 38/12; A61P 31/04

(52) U.S. Cl. CPC C07K 7/56; A61K 38/12; A61P 31/04

(57) ABSTRACT

Prodrugs of antibiotic teixobactin, methods of their synthesis, and methods of their use as antimicrobial treatments are described. The teixobactin prodrugs incorporate one or more O-acyl linkages. The O-acyl linkages can be utilized in substitution of amide peptide bonds between an amino acid containing an alcohol side chain and the immediately preceding amino acid.

### CROSS-REFERENCE TO RELATED APPLICATIONS

The current application claims priority to U.S. Provisional Patent Application No. 63/241,920, filed Sep. 8, 2021 and U.S. Provisional Patent Application No. 63/364,690, filed May 13, 2022; the disclosures of which are hereby incorporated by reference in their entireties.

### STATEMENT OF FEDERAL FUNDING

This invention was made with Government support under Grant No. AI156565 awarded by the National Institutes of Health. The Government has certain rights in the invention.

### SEQUENCE LISTING

The instant application contains a Sequence Listing which has been submitted electronically in ASCII format and is hereby incorporated by reference in its entirety. The ASCII copy was created on Sep. 8, 2022, is named 07482.xml, and is 40.8 KB in size.

### TECHNICAL FIELD

The present disclosure is related to antibiotic prodrugs, methods of synthesis and methods of treatment; and more particularly to prodrugs of teixobactin.

### BACKGROUND

Antimicrobial agents, including antibiotics, are used for the treatment or prevention of pathogenic bacterial infections. A major complication in the medical and health field, however, is antimicrobial resistance by pathogenic bacteria. The paucity of novel antimicrobial compounds exacerbates this problem, as treatments continue to utilize classical antibiotics.

Furthermore, antibiotic-resistant bacteria are becoming an even greater concern as the number of deaths due to these infections increases and antibiotic efficacy decreases. Gram-positive pathogens-including methicillin-resistant Staphylococcus aureus (MRSA), vancomycin-resistant Enterococci (VRE), and multidrug-resistant Mycobacterium tuberculosis (MDR-TB)—cause more than 45% of the deaths related to antibiotic resistant infections in the United States and more than 35% worldwide.

In 2015, Lewis and coworkers discovered teixobactin (FIG. 1; SEQ ID NO: 1), a non-ribosomal undecapeptide consisting of a linear tail and a macrolactone ring (L. L. Ling, et al., Nature 2015; 517:455-459, the disclosure of which is incorporated herein by reference.). Teixobactin inhibits gram-positive bacteria—including MRSA, VRE, and MDR-TB—by restricting peptidoglycan cell wall synthesis, a critical component for pathogen survival. As of yet, teixobactin resistant strains have not yet emerged due to their mechanism of inhibition that attacks the cell wall component, which is integral and conserved across all Gram-positive bacteria.

### SUMMARY

Embodiments of the disclosure are directed to antimicrobial teixobactin compounds and analogues thereof having O-acyl isopeptide linkages, methods of their synthesis, and methods of their administration. In several embodiments, a teixobactin compound or analogue has one or more O-acyl isopeptide linkages. In various embodiments, a teixobactin compound or analogue has an O-acyl isopeptide linkage between Ile₂ and Ser₃, between Ile₆ and Ser₇, or between both Ile₂ and Ser₃ and Ile₆ and Ser₇. In many embodiments, a teixobactin compound or analogue having one or more O-acyl isopeptide linkages is administered to a subject as prodrug, where the compound or analogue will undergo chemical alterations within the subject and the O-acyl isopeptide linkages will convert into amide peptide linkages and revert serine into a standard peptide configuration. Accordingly, in many embodiments, a teixobactin compound or analogue having one or more O-acyl linkages is administered to treat the subject for a bacterial infection, especially a gram-positive bacterial infection.

In one aspect, a peptide comprises a teixobactin peptide or a teixobactin analogue, or an enantiomer of the teixobactin peptide or the teixobactin analogue. The peptide has one or more O-acyl isopeptide linkages between two amino acids in place of an amide peptide bond.

In an aspect, the one or more O-acyl isopeptide linkages is capable of chemically converting into amide peptide bonds at physiological conditions.

In an aspect, the chemical conversion of the O-acyl isopeptide linkages results in one of the two amino acids to have an alcohol side chain.

In an aspect, the one or more O-acyl isopeptide linkages is between an amino acid with having a hydroxy group at the beta position and the immediately preceding amino acid.

In an aspect, the one or more O-acyl isopeptide linkages is between a serine and the immediately preceding amino acid.

In an aspect, the one or more O-acyl isopeptide linkages is between a threonine and the immediately preceding amino acid.

In an aspect, the one or more O-acyl isopeptide linkages is between an allo-threonine and the immediately preceding amino acid.

In an aspect, the one or more O-acyl isopeptide linkages is between a serine at position 7 and the immediately preceding amino acid.

In an aspect, the one or more O-acyl isopeptide linkages is between a serine at position 3 and the immediately preceding amino acid.

In an aspect, the one or more O-acyl isopeptide linkages is between a serine at position 7 and the immediately preceding amino acid and between a serine at position 3 and the immediately preceding amino acid.

In an aspect, the peptide includes Lys, Arg, Leu, or allo-End at position 10.

In an aspect, the peptide includes Thr or allo-Thr at residue position 7 or residue position 3, and wherein the one or more O-acyl isopeptide linkages is between the Thr or allo-Thr and the immediately preceding amino acid.

In an aspect, the peptide includes a hydrophobic D-amino acid with an NH₂, N-methyl or N-alkyl substituent at position 1.

In an aspect, the peptide includes a bulky hydrophobic amino acid at position 2, at position 6, or at position 11.

In an aspect, the peptide includes a basic amino acid at position 4 or at position 9.

In an aspect, the peptide includes a D-hydrophobic amino acid at position 5.

In an aspect, the peptide includes aza-D-Thr at position 8.

In an aspect, the peptide includes a macrolactam ring at residues 8 to 11.

In an aspect, a fluorophore is attached to one of the amino acids of the peptide.

In an aspect, the peptide sequence and structure are one of:

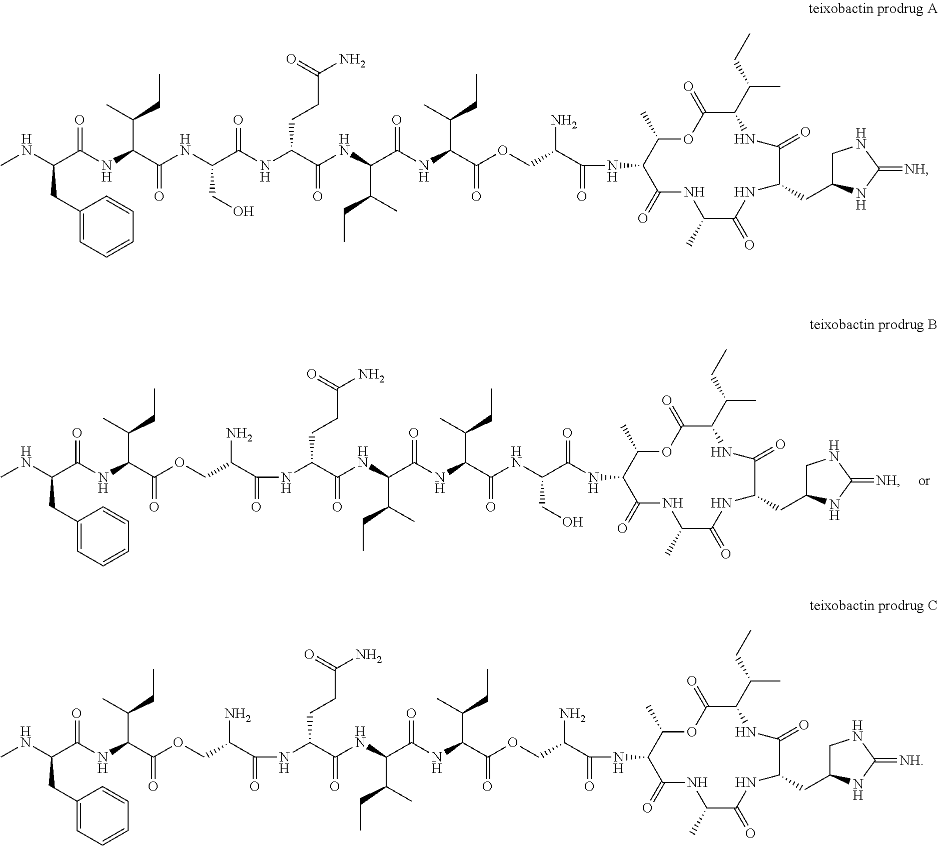

In an aspect, the peptide sequence and structure are one of:

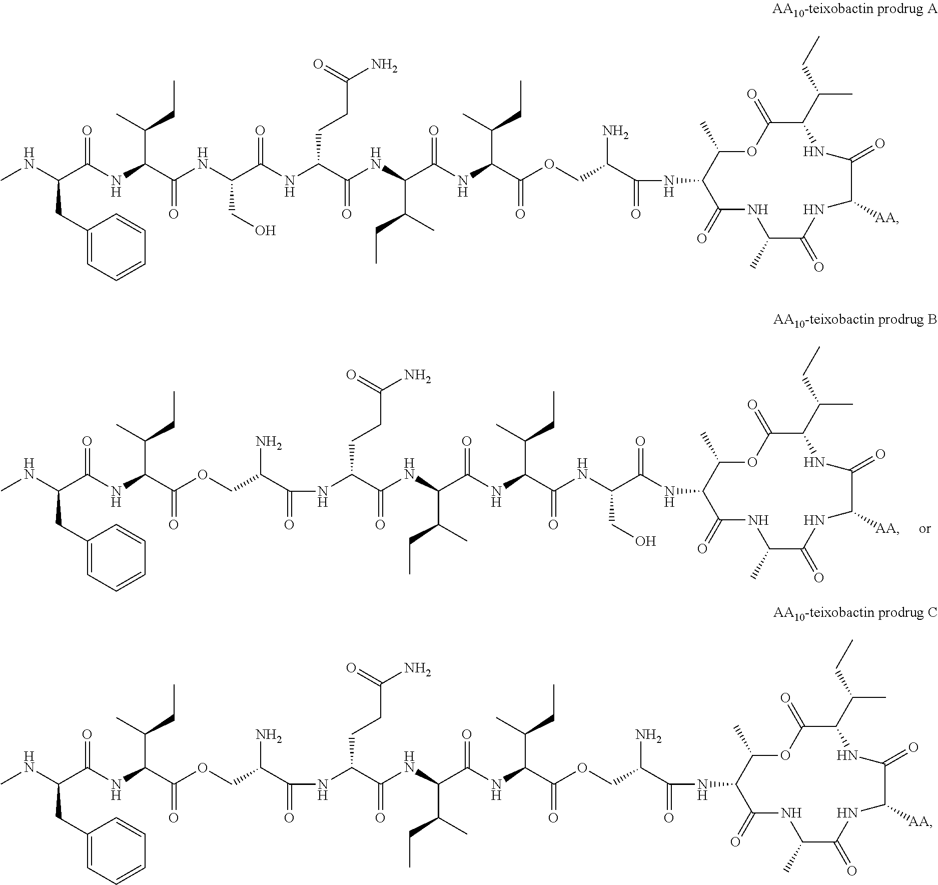

wherein AA is Lys, Arg, or Leu.

In an aspect, a medicament is for the treatment of an infection of gram-positive bacteria. The medicament comprises a teixobactin peptide or a teixobactin analogue, or an enantiomer of the teixobactin peptide or the teixobactin analogue. The peptide has one or more O-acyl isopeptide linkages between two amino acids in place of an amide peptide bond.

In an aspect, the medicament is formulated for oral, topical, ocular, transdermal, transmucosal, parentenal, intranasal, pulmonary, epicutaneous, subcutaneous, intramuscular, or intravenous administration.

In an aspect, a method is for treating an infection of gram-positive bacteria. The method comprises administering to a subject a teixobactin peptide or a teixobactin analogue, or an enantiomer of the teixobactin peptide or the teixobactin analogue. The peptide has one or more O-acyl isopeptide linkages between two amino acids in place of an amide peptide bond.

In an aspect, the teixobactin peptide or the teixobactin analogue, or an enantiomer of the teixobactin peptide or the teixobactin analogue, is orally, topically, ocularly, transdermally, transmucosally, parentenally, intranasally, pulmonarily, epicutaneously, subcutaneously, intramuscularly, or intravenously administered.

In an aspect, teixobactin peptide or a teixobactin analogue, or an enantiomer of the teixobactin peptide or the teixobactin analogue, having one or more O-acyl isopeptide linkages between two amino acids in place of an amide peptide bond is for the treatment of an infection of gram-positive bacteria.

In an aspect, the treatment comprises an administration that is oral, topical, ocular, transdermal, transmucosal, parentenal, intranasal, pulmonari, epicutaneous, subcutaneous, intramuscular, or intravenous.

### BRIEF DESCRIPTION OF THE DRAWINGS

These and other features and advantages of the present invention will be better understood by reference to the following detailed description when considered in conjunction with the accompanying data and figures, wherein:

FIG. 1 provides a chemical structure diagram of teixobactin in accordance with the prior art. SEQ ID No: 1.

FIGS. 2A to 2C provide chemical structure diagrams of O-acyl isopeptide teixobactin in accordance with various embodiments. FIG. 2A incorporates an O-acyl isopeptide linkage between Ile₆ and Ser₇. FIG. 2B incorporates an O-acyl isopeptide linkage between Ile₂ and Ser₃. FIG. 2C incorporates an O-acyl isopeptide linkage between Ile₆ and Ser₇ and between Ile₂ and Ser₃. SEQ ID Nos: 2-4.

FIGS. 3A to 3C provide chemical structure diagrams of O-acyl isopeptide AA₁₀-teixobactin in accordance with various embodiments. FIG. 3A incorporates an O-acyl isopeptide linkage between Ile₆ and Ser₇. FIG. 3B incorporates an O-acyl isopeptide linkage between Ile₂ and Ser₃. FIG. 3C incorporates an O-acyl isopeptide linkage between Ile₆ and Ser₇ and between Ile₂ and Ser₃. SEQ ID Nos: 5-13.

FIG. 4 provides a reaction mechanism of converting O-acyl isopeptide linkages into amide peptide linkages in accordance with various embodiments.

FIG. 5 provides a schematic for synthesizing O-acyl isopeptide Lys₁₀-teixobactin prodrug A in accordance with various embodiments.

FIGS. 6A to 6H provide conversion data of O-acyl isopeptide teixobactin analogues into amide peptide teixobactin analogues in accordance with various embodiments. FIG. 6A provides analytical RP-HPLC trace showing clean conversion of Lys₁₀-teixobactin prodrug A to Lys₁₀-teixobactin. FIGS. 6B, 6C and 6D provide conversion kinetics of Lys₁₀-teixobactin prodrugs (FIG. 6B), Arg₁₀-teixobactin prodrugs (FIG. 6C), and Leu₁₀-teixobactin prodrugs (FIG. 6D). FIGS. 6E, 6F and 6G provide conversion percentages of Lys₁₀-teixobactin prodrug C (FIG. 6E), Arg₁₀-teixobactin prodrug C (FIG. 6F), and Leu₁₀-teixobactin prodrug C (FIG. 6G). FIG. 6H provides a summary table of the half-lives of O-acyl isopeptide teixobactin analogues.

FIG. 7 provides a table of MIC values of teixobactin O-acyl isopeptide prodrug analogues in accordance with various embodiments.

FIGS. 8A, 8B and 8C provide gelation results teixobactin O-acyl isopeptide prodrug analogues in accordance with various embodiments. FIG. 8A provides gelation assay results of Lys₁₀-teixobactin O-acyl isopeptide prodrugs. FIG. 8B provides gelation assay results of Arg₁₀-teixobactin O-acyl isopeptide prodrugs. FIG. 8C provides gelation assay results of Leu₁₀-teixobactin O-acyl isopeptide prodrugs.

FIGS. 9A to 9H provide hemolytic assay results of teixobactin O-acyl isopeptide prodrug analogues in accordance with various embodiments. FIGS. 9A and 9B provide hemolytic assay results of Lys₁₀-teixobactin O-acyl isopeptide prodrug analogues without polysorbate 80 (FIG. 9A) and with polysorbate 80 (FIG. 9B). FIGS. 9C and 9D provide hemolytic assay results of Arg₁₀-teixobactin O-acyl isopeptide prodrug analogues without polysorbate 80 (FIG. 9C) and with polysorbate 80 (FIG. 9D). FIGS. 9E and 9F provide hemolytic assay results of Leu₁₀-teixobactin O-acyl isopeptide prodrug analogues without polysorbate 80 (FIG. 9E) and with polysorbate 80 (FIG. 9F). FIGS. 9G and 9H provide hemolytic assay results of teixobactin an vancomycin without polysorbate 80 (FIG. 9G) and with polysorbate 80 (FIG. 9H).

FIGS. 10A to 10F provide cytotoxicity assay results of teixobactin O-acyl isopeptide prodrug analogues in accordance with various embodiments. FIG. 10A provides hemolytic assay results of Lys₁₀-teixobactin O-acyl isopeptide prodrug A and of Lys₁₀-teixobactin O-acyl isopeptide prodrug B. FIG. 10B provides hemolytic assay results of Lys₁₀-teixobactin O-acyl isopeptide prodrug C and of Lys₁₀-teixobactin. FIG. 10C provides hemolytic assay results of Arg₁₀-teixobactin O-acyl isopeptide prodrug A and of Arg₁₀-teixobactin O-acyl isopeptide prodrug B. FIG. 10D provides hemolytic assay results of Arg₁₀-teixobactin O-acyl isopeptide prodrug C and of Arg₁₀-teixobactin. FIG. 10E provides hemolytic assay results of Leu₁₀-teixobactin O-acyl isopeptide prodrug A and of Leu₁₀-teixobactin O-acyl isopeptide prodrug B. FIG. 10F provides hemolytic assay results of Leu₁₀-teixobactin O-acyl isopeptide prodrug C and of Leu₁₀-teixobactin.

### DETAILED DISCLOSURE

Turning now to the diagrams and figures, teixobactin prodrugs, their synthesis and use as antimicrobial treatments are described. In some embodiments, antimicrobial prodrug compounds are teixobactin and teixobactin analogues having one or more O-acyl isopeptide linkages. These compounds are referred to herein as O-acyl isopeptide teixobactin or O-acyl isopeptide teixobactin analogues. For sake of simplicity, the chemistry, structures and properties of the O-acyl isopeptide compounds are described throughout the description with reference to teixobactin and a few analogues, however, it is to be understood that the various embodiments of the disclosure include any analogue of teixobactin capable of incorporating one or more O-acyl isopeptide linkages.

In various embodiments, an O-acyl isopeptide teixobactin has an O-acyl isopeptide linkage between Ile₂ and Ser₃, between Ile₆ and Ser₇, or between Ile₂ and Ser₃ and Ile₆ and Ser₇. Experimentation with O-acyl isopeptide teixobactin has shown that these compounds have improved chemical and physical properties as compared to their standard amide peptide teixobactin counterparts. Furthermore, O-acyl isopeptide teixobactin is capable of converting into amide peptide teixobactin within a treated individual, meaning the isopeptide linkages convert into amide peptide linkages that are standard in naturally derived teixobactin within the individual after administration of the compound. Accordingly, teixobactin with O-acyl isopeptide linkages can be utilized as a prodrug and administered to an individual for antimicrobial treatment. In many embodiments, antibiotic therapeutics comprise antimicrobial O-acyl isopeptide teixobactin and analogues, which can be utilized to treat an individual having a bacterial infection. Teixobactin has strong antimicrobial activity against gram-positive bacteria including most (if not all) drug-resistant bacteria. Based on this activity, in accordance with several embodiments, O-acyl isopeptide teixobactin is administered to treat an individual with a gram-positive pathogen infection, including (but not limited to) to infections of Staphylococcus, Streptococcus, Clostridium, Bacillus, Corynebacterium, and Listeria. Furthermore, in accordance with many embodiments, O-acyl isopeptide teixobactin is administered to treat an individual with an infection of difficult-to-treat pathogens such as (for example) Bacillus anthracis (Anthrax), Staphylococcus aureus (MRSA), vancomycin-resistant Enterococci (VRE), and multidrug-resistant Mycobacterium tuberculosis (MDR-TB).

The various embodiments provided within the disclosure are based on the finding that O-acyl isopeptide teixobactin and O-acyl isopeptide analogues improve solubility and antimicrobial activity as compared to amide peptide teixobactin. Generally, amide peptide teixobactin and amide peptide analogues can aggregate, coagulate and/or precipitate in aqueous solutions, forming viscous gels. This aggregative property can cause problems when administered to an individual for a multitude of reasons. Aggregation of amide peptide teixobactin may prevent the compound from systemically dispersing throughout the subject's body and thus prevent the compound from adequately reaching the pathogenic infection. Furthermore, and perhaps more concerning is the possibility that coagulation and/or precipitation of amide peptide teixobactin may result in formation of an embolus when administered to a subject and may cause an embolism in the subject's vasculature, lungs, brains, heart, or other area of the body. Because of this property, the dosage of amide peptide teixobactin for administration must be maintained at a concentration that prevents aggregation. Because O-acyl isopeptide teixobactin and O-acyl isopeptide analogues have greatly decreased aggregation, and in accordance with several embodiments, O-acyl isopeptide teixobactin and O-acyl isopeptide analogues can be administered to a subject at higher doses than amide peptide teixobactin.

Throughout the description and claims, the term “residue position” is in reference to the teixobactin peptide as isolated from its natural source. Accordingly, the residue position of various embodiments of teixobactin peptides, teixobactin analogues, and/or enantiomers of teixobactin peptides or teixobactin analogues is based on the position of the natural teixobactin peptide regardless of whether an insertion or a deletion is present in the peptide. For example, various embodiments are described to have an O-acyl isopeptide linkage at residue position 3 and/or at residue position 7, which is a substitution of the naturally occurring amide peptide linkage of a Ser₃ and/or Ser₇ of the naturally occurring teixobactin peptide. For simplicity and consistency, the various embodiments of peptides having one or more of these substitutions are described and claimed throughout as occurring at residue position 3 and/or at residue position 7 regardless of whether a particular embodiment includes an insertion or deletion that would alter the actual position of the substituted amino acid. Furthermore, the description and claims of embodiments of the disclosure can include insertions and deletions even when the description or claim is described as having a particular amino acid at a particular residue position.

### Structure and Function of Teixobactin

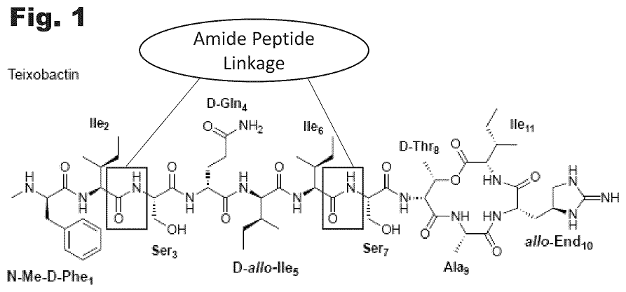

The antibiotic teixobactin, first reported in 2015, is a nonribosomal undecapeptide containing a macrocyclic depsipeptide group (FIG. 1; SEQ ID NO: 1). As such, it contains four D-amino acids and seven L-amino acids, wherein the C-terminal's Ile₁ is cyclized onto the side chain of D-Thr₈ to form a 13-membered lactone. In addition, residue 10 of teixobactin is a rare nonproteinogenic amino acid, L-allo-enduracididine (allo-End₁₀), which is a cyclic analogue of arginine. Notably, each of the linkages between the amino acids (except the cyclization of Ile₁₁ to D-Thr₈) is a standard amide peptide linkage. Highlighted in boxes in FIG. 1 are the amide peptide linkages between Ile₂ and Ser₃ and Ile₆ and Ser₇; these amide peptide linkages are modified in various embodiments of compounds described herein.

It has been postulated that, similar to the action mechanism of known antibiotic vancomycin, teixobactin's efficacy stems from its ability to inhibit cell wall formation in Gram-positive bacteria by binding to the wall's lipid II (via prenyl-pyrophosphate-GlcNAc region) and related peptidoglycan precursors. Therefore, since these binding targets are highly conserved in bacteria and cannot easily mutate to impart drug resistance, teixobactin-based antibiotics offer great promise to the efforts directed against rising resistance in pathogens such as methicillin-resistant Staphylococcus aureus (MRSA).

### O-Acyl Isopeptide Teixobactin and Analogues

Several embodiments are directed to teixobactin and teixobactin analogues having one or more O-acyl isopeptide linkages. Any teixobactin analogue having an amino acid with a sidechain having a hydroxy group at the beta position can be utilized to formulate an O-acyl isopeptide linkage, where the O-acyl isopeptide linkage is between the amino acid with the hydroxy group side chain and the immediately preceding amino acid. Naturally occurring standard proteinogenic amino acids having a hydroxy group at the beta position include serine, threonine, and allo-threonine and thus can be utilized to formulate O-acyl isopeptide linkage with an immediately preceding amino acid. Any amino acid with a hydroxy group at the beta position, whether naturally occurring or not, or whether it is a standard proteinogenic amino acid or not, can be utilized to formulate O-acyl isopeptide linkage. As shown in FIG. 1, teixobactin naturally includes two amino acids with available beta hydroxy groups in Ser₃ and Ser₇. Accordingly, several embodiments are directed to forming O-acyl isopeptide linkages in teixobactin or any analogue utilizing Ser₃, Ser₇, or both Ser₃ and Ser₇. Various analogues of teixobactin can incorporate one or more amino acids (in addition to or in lieu of Ser₃ and Ser₇) having a beta hydroxy group in the side chain into the macromolecule and thus these analogues are contemplated with the various embodiments of the description. Furthermore, any analogue that results in a change of residue position of Ser₃ or Ser₇ are also contemplated with the various embodiments of the description (e.g., inserting or deleting one or more amino acids into teixobactin such that either Ser₃ or Ser₇ is in a different residue position than 3 or 7).

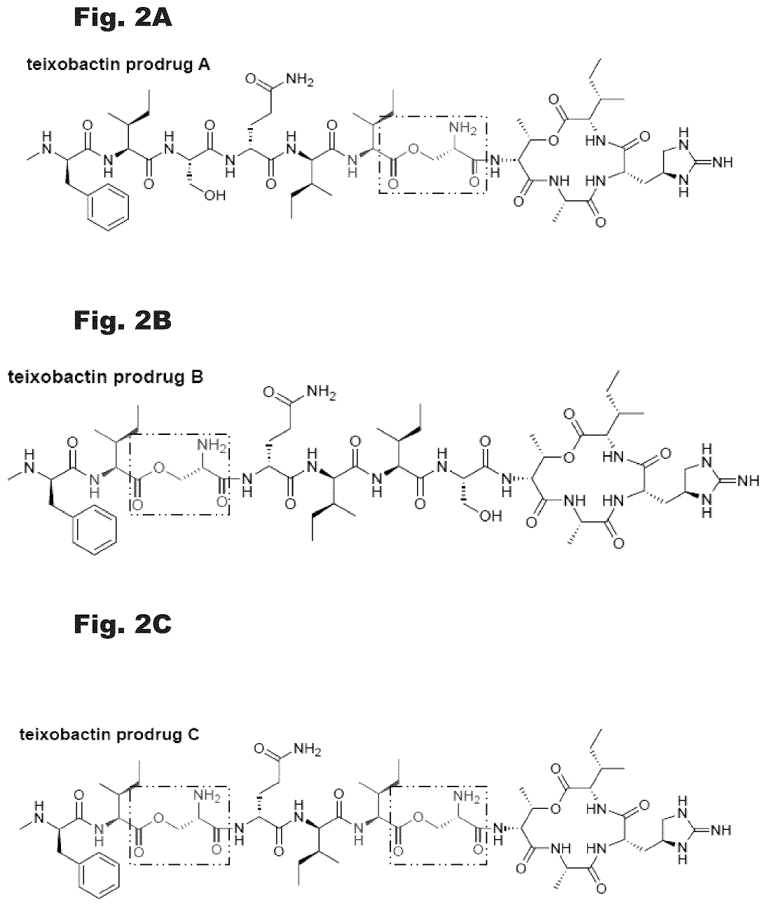

Provided in FIGS. 2A to 2C are examples of O-acyl isopeptide teixobactin having an O-acyl isopeptide linkage between Ile₆ and Ser₇ (FIG. 2A; SEQ ID NO: 2), an O-acyl isopeptide linkage between Ile₂ and Ser₃ (FIG. 2B; SEQ ID NO: 3), and an O-acyl isopeptide linkage between both Ile₂ and Ser₃, and Ile₆ and Ser₇ (FIG. 2C; SEQ ID NO: 4). The O-acyl isopeptide linkages are highlighted in the structure diagrams utilizing rectangular boxes.

As can be seen in these figures, the O-acyl isopeptide linked serine has an altered structure such that the beta hydroxy group of the side chain is utilized to form the O-acyl isopeptide with the carbonyl of the immediately preceding amino acid. The O-acyl isopeptide linkage results in the serine having a free amine. In several embodiments, the free amine is protonated to form a salt with any anion. In some embodiments, the anion is an anion commonly utilized in pharmaceutical compounds. Anions that can be utilized include (but are not limited to) acetate, benzenesulfonate, benzoate, bromide, chloride, citrate, fluoride, formate, fumarate, gluconate, glucuronate, glutarate, glycolate, lactate, malate, malonate, maleate, nitrate, nitrite, phosphate, pyruvate, succinate, sulfate, tartrate, tosylate, and trifluoroacetate.

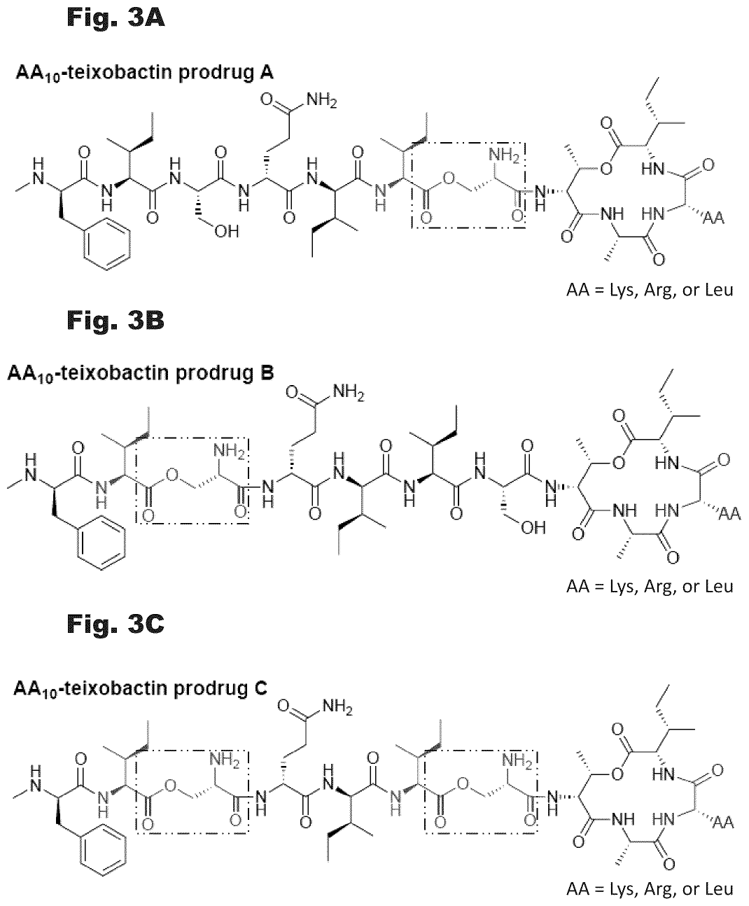

Provided in FIGS. 3A to 3C are examples of O-acyl isopeptide teixobactin analogues having one or two O-acyl isopeptide linkage(s) and a substituted amino acid at position 10. Specifically, FIGS. 3A to 3C provide O-acyl isopeptide teixobactin analogues having Lys₁₀, Arg₁₀, or Leu₁₀ in place of the naturally occurring allo-End₁₀ and an O-acyl isopeptide linkage between Ile₆ and Ser₇ (FIG. 3A; SEQ ID NO: 5, 8, or 11), an O-acyl isopeptide linkage between Ile₂ and Ser₃ (FIG. 3B; SEQ ID NO: 6, 9 or 12), and an O-acyl isopeptide linkage between both Ile₂ and Ser₃, and Ile₆ and Ser₇ (FIG. 3C; SEQ ID NO: 7, 10, or 13).

Numerous other O-acyl isopeptide teixobactin analogues are contemplated. Various embodiments incorporate one or more substitutions. A substitution is a change of amino acid, a structural alteration of amino acid, an insertion of an amino acid, or a deletion of an amino acid. In some embodiments, an analogue of O-acyl isopeptide teixobactin incorporates at least one substitution. In some embodiments, an analogue of O-acyl isopeptide teixobactin incorporates at least two substitutions. In some embodiments, an analogue of O-acyl isopeptide teixobactin incorporates at least three substitutions. In some embodiments, an analogue of O-acyl isopeptide teixobactin incorporates at least four substitutions. In some embodiments, an analogue of O-acyl isopeptide teixobactin incorporates at least five substitutions. In some embodiments, an analogue of O-acyl isopeptide teixobactin incorporates at least six substitutions. In some embodiments, an analogue of O-acyl isopeptide teixobactin incorporates at least seven substitutions. In some embodiments, an analogue of O-acyl isopeptide teixobactin incorporates at least eight substitutions. Any substitution described herein can be combined with any other substitution(s) described herein that still yield a teixobactin analogue.

In various embodiments, allo-End₁₀ is substituted with any other amino acid.

In various embodiments, Ser₇ is substituted with threonine, where the threonine can be O-acyl isopeptide linked or amide peptide linked with the immediately preceding amino acid. In various embodiments, Ser₃ is substituted with threonine, where the threonine can be O-acyl isopeptide linked or amide peptide linked with the immediately preceding amino acid. In various embodiments, Ser₇ is substituted with allo-threonine, where the allo-threonine can be O-acyl isopeptide linked or amide peptide linked with the immediately preceding amino acid. In various embodiments, Ser₃ is substituted with allo-threonine, where the allo-threonine can be O-acyl isopeptide linked or amide peptide linked with the immediately preceding amino acid. In some embodiments, Ser₇ is substituted with an amino acid having a beta hydroxy group, where the amino acid having a beta hydroxy group can be O-acyl isopeptide linked or amide peptide linked with the immediately preceding amino acid. In some embodiments, Ser₃ is substituted with an amino acid having a beta hydroxy group, where the amino acid having a beta hydroxy group can be O-acyl isopeptide linked or amide peptide linked with the immediately preceding amino acid.

In various embodiments, N-Me-D-Phe₁ is substituted with any other hydrophobic amino acid. In various embodiments, N-Me-D-Phe₁ is substituted with any other hydrophobic D-amino acid with an NH₂, N-methyl or N-alkyl substituent. In various embodiments, N-Me-D-Phe₁ is substituted with Phe.

In various embodiments, Ile₂ is substituted with any other hydrophobic amino acid. In various embodiments, Ile₂ is substituted with any other bulky hydrophobic amino acid. A bulky hydrophobic amino acid is any amino acid with a side chain that promotes hydrophobic interactions. Examples of bulky amino acids include (but are not limited to) Val, Leu, lie and cyclohexylglycine (Chg).

In various embodiments, D-Gln₄ is substituted with any other amino acid. In various embodiments, D-Gln₄ is substituted with any other D-amino acid. In various embodiments, D-Gln₄ is substituted with a basic amino acid. In various embodiments, D-Gln₄ is substituted with Arg, Lys, or Orn.

In various embodiments, D-allo-Ile₅ is substituted with any other hydrophobic amino acid. In various embodiments, D-allo-Ile₅ is substituted with any other D-hydrophobic amino acid.

In various embodiments, Ile₆ is substituted with any other hydrophobic amino acid. In various embodiments, Ile₆ is substituted with any other bulky hydrophobic amino acid. A bulky hydrophobic amino acid is any amino acid with a side chain that promotes hydrophobic interactions. Examples of bulky amino acids include (but are not limited to) Val, Leu, lie and cyclohexylglycine (Chg).

In various embodiments, D-Thr₈ is substituted with a variant of D-Thr₈. In various embodiments, D-Thr₈ is substituted with aza-D-Thr₈.

In various embodiments, Alas is substituted with any other amino acid. In various embodiments, Alas is substituted with a basic amino acid. In various embodiments, Ala₉ is substituted with Arg, Lys, or Orn.

In various embodiments, Ile₁₁ is substituted with any other hydrophobic amino acid. In various embodiments, Ile₁₁ is substituted with any other bulky hydrophobic amino acid. A bulky hydrophobic amino acid is any amino acid with a side chain that promotes hydrophobic interactions. Examples of bulky amino acids include (but are not limited to) Val, Leu, lie and cyclohexylglycine (Chg).

In various embodiments, the macrolactone ring (i.e, residues 8-11) is replaced with a macrolactam ring, in which D-Thr₈ is replaced with a nitrogen-containing analogue, such as (for example) 2,3-diaminobutanoic acid.

Various embodiments are directed towards enantiomers O-acyl isopeptide teixobactin and analogues. It has been found that enantiomer conformations of teixobactin have similar antibiotic activity to their standard counterpart (see, e.g., H. Yang, K. H. Chen, and J. S. Nowick, ACS Chem Biol. 2016; 11:1823-6, the disclosure of which is incorporated herein by reference).

In various embodiments, one or more of the hydrogens of the O-acyl isopeptide teixobactin or analogue is deuterated. It has been found that deuterated analogs of prodrugs have improved stability, which may result in a smaller concentration or less frequent dosing regimen.

Various embodiments are directed to further substitutions of teixobactin and teixobactin analogues. Many examples of substitutions of teixobactin and teixobactin analogues have been described in art (see, e.g., J. A. Karas, et al., Ann N Y Acad Sci. 2020; 1459:86-105; and H. Yang, et al., Chem Commun (Camb). 2017; 53:2772-2775; the disclosures of which are each incorporated herein by reference).

In various embodiments, a fluorophore is attached to one of the amino acids of the O-acyl isopeptide teixobactin or analogue. In various embodiments, a fluorophore is attached to residue 10 (e.g., allo-End₁₀, Lys₁₀, or Arg₁₀). Adding a fluorophore allows the O-acyl isopeptide teixobactin or analogue to be monitored by fluorescence, which can be performed by any fluorescent methodology, including (but not limited to) microscopy, cytometry, and in vivo fluorescence imaging.

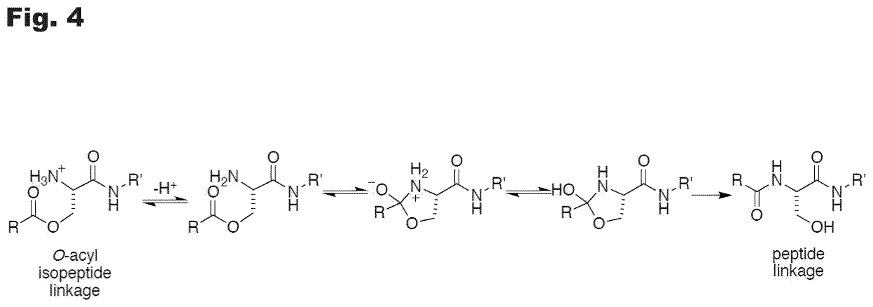

In several embodiments, the O-acyl isopeptide teixobactin or analogue converts its O-acyl isopeptide linkages into standard amide peptide linkages in the individual when administered (typically conversion occurs in the bloodstream). Provided in FIG. 4 is molecular reaction diagram depicting the nucleophilic acyl substitution reaction to convert O-acyl isopeptide linkages into standard amide peptide linkages. As can be seen in the figure, the free amine of the O-acyl isopeptide linked serine serves a nucleophile and forms a bond with the carbonyl of the O-acyl isopeptide linkage to form a transitory ring structure. The oxygen of the ester isopeptide linkage serves as the leaving group, which converts into an alcohol to generate the alcohol side group of serine. The reaction results in a standard amide peptide linkage between the serine and the immediately preceding amino acid.

### Therapeutic Formulations & Modes of Treatment

Several embodiments are directed to the use of O-acyl isopeptide teixobactin or an O-acyl isopeptide teixobactin analogue as a prodrug compound within a therapeutic for the treatment of a bacterial infection, especially an infection of gram-positive bacteria. As described herein, O-acyl isopeptide teixobactin or an O-acyl isopeptide teixobactin analogue converts its O-acyl isopeptide linkages in an individual to formulate the active teixobactin or analogue compound. In some embodiments, O-acyl isopeptide teixobactin or an O-acyl isopeptide teixobactin analogue are administered in a therapeutically effective amount to an individual as part of a course of treatment. Individuals are to include any animal, including (but not limited to) an animal, a mammal, a bird, a reptile, a primate, a human, a pet, a farm animal, or a zoo animal. As used in this context, to “treat” means to ameliorate at least one symptom of the disorder to be treated or to provide a beneficial physiological effect. For compounds for the treatment of bacterial infection, amelioration of a symptom could be inhibition of bacteria replication or reduction of one or more symptoms related to the infection of the pathogen. Assessment of amelioration can be performed in many ways, including, but not limited to assessing active bacterial infection (e.g., plate streaking), or reduction in pathogen related symptoms.

Numerous embodiments are directed to the use of O-acyl isopeptide teixobactin or an O-acyl isopeptide teixobactin analogue as a prodrug antibiotic. In several embodiments, an individual to be treated is infected with bacteria or is at risk of bacterial infection (e.g., prophylactic treatment). A number of bacterial pathogens can be treated. In some embodiments, O-acyl isopeptide teixobactin or an O-acyl isopeptide teixobactin analogue is administered to treat an individual with an infection of or at risk of being infected by a gram-positive pathogen, including (but not limited to) to infections of Staphylococcus, Streptococcus, Clostridium, Bacillus, Corynebacterium, and Listeria. Various pathogenic Staphylococcus that can be treated include (but are not limited to) S. aureus and S. epidermidis. Various pathogenic Streptococcus that can be treated include (but are not limited to) S. pneumoniae, S. pyogenes and S. agalactiae. Various pathogenic Clostridium that can be treated include (but are not limited to) C. tetani, C. botulinum, C. perfringens and C. difficile. Various pathogenic Bacillus that can be treated include (but are not limited to) B. anthracis and B. cereus. Various pathogenic Corynebacterium that can be treated include (but are not limited to) C. diphtheria. Various pathogenic Listeria that can be treated include (but are not limited to) L. monocytogenes. Furthermore, in accordance with some embodiments, O-acyl isopeptide teixobactin is administered to treat an individual with an infection of difficult-to-treat pathogens such as (for example) Bacillus anthracis (Anthrax), Staphylococcus aureus (MRSA), vancomycin-resistant Enterococci (VRE), and multidrug-resistant Mycobacterium tuberculosis (MDR-TB).

A therapeutically effective amount can be an amount sufficient to prevent reduce, ameliorate or eliminate the symptoms of diseases or pathological conditions susceptible to such treatment. In some embodiments, a therapeutically effective amount is an amount sufficient to inhibit bacterial replication in an infected individual or mitigate infection in a prophylactic treatment.

Dosage, toxicity and therapeutic efficacy of the compounds can be determined, e.g., by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to non-infected cells. In many embodiments, therapeutic formulations comprising O-acyl isopeptide teixobactin or analogues are administered at a greater dosage concentration than therapeutic formulations comprising amide peptide teixobactin or analogues, in which dosage concentration may be limited due to aggregation.

Data obtained from cell culture assays or animal studies can be used in formulating a range of dosage for use in a subject. If the medicament is provided systemically, the dosage of such compounds lies preferably within a range of circulating concentrations that include the ED₅₀ with little or no toxicity. The dosage may vary within this range depending upon the dosage form employed and the route of administration utilized. For any compound used in a method of the various embodiments, the therapeutically effective dose can be estimated initially from cell culture assays. A dose may be formulated in animal models to achieve a circulating plasma concentration or within the local environment to be treated in a range that includes the IC₅₀ (i.e., the concentration of the test compound that achieves a half-maximal inhibition of bacterial replication) as determined in cell culture. Such information can be used to more accurately determine useful doses in humans. Levels in plasma may be measured, for example, by liquid chromatography coupled to mass spectrometry. In some embodiments, a therapeutic effect is achieved with an IC₅₀ less than 2.0 μg/mL, less than 1.5 μg/mL, less than 1.0 μg/mL, less than 0.5 μg/mL, less than 0.25 μg/mL, or less than 0.125 μg/mL.

An “effective amount” is an amount sufficient to effect beneficial or desired results. For example, a therapeutic amount is one that achieves the desired therapeutic effect. This amount can be the same or different from a prophylactically effective amount, which is an amount necessary to mitigate and/or prevent onset of disease or disease symptoms. An effective amount can be administered in one or more administrations, applications or dosages. A therapeutically effective amount of a composition depends on the composition selected. The compositions can be administered one or more times per day, or one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. For example, several divided doses may be administered daily, one dose, or cyclic administration of the compounds to achieve the desired therapeutic result. A single peptide compound of a particular formula may be administered, or combinations of various peptide compounds encompassing multiple formulas may be administered.

In a number of embodiments, peptide compounds are administered in combination with an appropriate standard of care, such as the standard of care established by an appropriate agency (e.g., the United States Federal Drug Administration (FDA)). In many embodiments, peptide compounds are administered in combination with other antibiotic or immune stimulatory compounds, especially agency-approved compounds.

A number of embodiments of formulations of O-acyl isopeptide teixobactin and analogues are suggested for oral, topical, ocular, transdermal, transmucosal, parentenal, intranasal, pulmonary, epicutaneous, subcutaneous, intramuscular, or intravenous administration, as determined by various factors including compound bioavailability and the pathogen to be treated. Formulations may conveniently be presented in unit dosage form and may be prepared by any of the methods known in the art of pharmacy. Typically, these methods include the step of bringing into association a compound of at least one embodiment described herein, or a pharmaceutically acceptable salt or solvate thereof with the carrier which constitutes one or more accessory ingredients. An ocular drug delivery system may be utilized for delivery via ocular drops, ocular mist, ocular ointment, ocular gel, ocular cream, or any other appropriate system known in the art of pharmacy. An intranasal drug delivery system may be utilized for delivery via a nasal spray, nasal mist, nebulizer, or any other appropriate system known in the art of pharmacy. Likewise, a pulmonary drug delivery system may be utilized, for aerosol delivery via an inhaler, a nebulizer, dry powder, or any other appropriate system known in the art of pharmacy. An epicutaneous drug delivery system may be utilized for topical delivery via a cream, foam, gel, lotion, ointment, medicated patch, or any other appropriate system known in the art of pharmacy. A subcutaneous drug delivery system may be utilized for delivery via a syringe or any other appropriate system known in the art of pharmacy. An intramuscular drug delivery system may be utilized for delivery via a syringe or any other appropriate system known in the art of pharmacy. Further, an intravenous drug delivery system may be utilized, for fluid delivery via a syringe, infusion (e.g., drips), or any other appropriate system known in the art of pharmacy.

Various agents can be incorporated that improve the solubility of the various compounds described herein. For example, various compounds can be formulated with one or more adjuvants and/or pharmaceutically acceptable carriers according to the selected route of administration. For oral applications, gelatin, flavoring agents, or coating material can be added. In general, for solutions or emulsions, including for use in pulmonary applications, carriers may include aqueous or alcoholic/aqueous solutions, emulsions or suspensions, including saline and buffered media. Parenteral vehicles can include sodium chloride and potassium chloride, among others. In addition, intravenous vehicles can include fluid and nutrient replenishers, electrolyte replenishers and the like.

For IV applications, numerous fluids can be utilized in accordance with various embodiments. In some embodiments, a crystalloid or colloid solution is utilized. Crystalloid solutions include (but are not limited to) saline (i.e., NaCl 0.9%), lactated Ringer's, and Ringer's acetate. Colloid solutions include (but are not limited to) blood, albumin, and plasma. Medications can be administered in a continuous infusion, a secondary infusion, or a bolus is utilized.

For oral applications, numerous coating agents can be used in accordance with various embodiments. In some embodiments, the coating agent is one which acts as a coating agent in conventional delayed release oral formulations, including polymers for enteric coating. Examples include hypromellose phthalate (hydroxy propyl methyl cellulose phthalate; HPMCP); hydroxypropylcellulose (HPC; such as KLUCEL®); ethylcellulose (such as ETHOCEL®); and methacrylic acid and methyl methacrylate (MAA/MMA; such as EUDRAGIT®).

Various embodiments of formulations also include at least one disintegrating agent, as well as diluent. In some embodiments, a disintegrating agent is a super disintegrant agent. One example of a diluent is a bulking agent such as a polyalcohol. In many embodiments, bulking agents and disintegrants are combined, such as, for example, PEARLITOL FLASH®, which is a ready to use mixture of mannitol and maize starch (mannitol/maize starch). In accordance with a number of embodiments, any polyalcohol bulking agent can be used when coupled with a disintegrant or a super disintegrant agent. Additional disintegrating agents include, but are not limited to, agar, calcium carbonate, maize starch, potato starch, tapioca starch, alginic acid, alginates, certain silicates, and sodium carbonate. Suitable super disintegrating agents include (but are not limited to) crospovidone, croscarmellose sodium, AMBERLITE (Rohm and Haas, Philadelphia, Pa.), and sodium starch glycolate.

In certain embodiments, diluents are selected from the group consisting of mannitol powder, spray dried mannitol, microcrystalline cellulose, lactose, dicalcium phosphate, tricalcium phosphate, starch, pregelatinized starch, compressible sugars, silicified microcrystalline cellulose, and calcium carbonate.

Several embodiments of a formulation further utilize other components and excipients. For example, sweeteners, flavors, buffering agents, and flavor enhancers to make the dosage form more palatable. Sweeteners include, but are not limited to, fructose, sucrose, glucose, maltose, mannose, galactose, lactose, sucralose, saccharin, aspartame, acesulfame K, and neotame. Common flavoring agents and flavor enhancers that may be included in the formulation of the present invention include, but are not limited to, maltol, vanillin, ethyl vanillin, menthol, citric acid, fumaric acid, ethyl maltol and tartaric acid.

Multiple embodiments of a formulation also include a surfactant. In certain embodiments, surfactants are selected from the group consisting of Tween 80, sodium lauryl sulfate, and docusate sodium.

Many embodiments of a formulation further utilize a binder. In certain embodiments, binders are selected from the group consisting of povidone (PVP) K29/32, hydroxypropylcellulose (HPC), hydroxypropylmethylcellulose (HPMC), ethylcellulose (EC), corn starch, pregelatinized starch, gelatin, and sugar.

Various embodiments of a formulation also include a lubricant. In certain embodiments, lubricants are selected from the group consisting of magnesium stearate, stearic acid, sodium stearyl fumarate, calcium stearate, hydrogenated vegetable oil, mineral oil, polyethylene glycol, polyethylene glycol 4000-6000, talc, and glyceryl behenate.

Preservatives and other additives, like antimicrobial, antioxidant, chelating agents, and inert gases, can also be present. (See generally, Remington's Pharmaceutical Sciences, 16th Edition, Mack, (1980), the disclosure of which is incorporated herein by reference.)

### EXEMPLARY EMBODIMENTS

Biological data supports the biological activity and medicinal applications of the aforementioned O-acyl isopeptide teixobactin and analogues. Described within are details of various O-acyl isopeptide teixobactin analogues designed to be a prodrug and elicit an inhibitory response on gram-positive bacteria. Data results are also provided. The data results of examples provided suggest that O-acyl isopeptide prodrugs undergo clean conversion to the corresponding teixobactin analogues at physiological pH. The prodrugs exhibit comparable or slightly improved antibiotic activity to the corresponding amide-linked teixobactin analogues. The prodrugs also exhibit improved solubility in aqueous conditions and do not gelate immediately upon exposure to physiological conditions. Hemolytic assays with human red blood cells show little to no hemolytic activity, and cytotoxicity assays with HeLa cells show no significant cytotoxicity. These findings suggest that teixobactin prodrugs and teixobactin prodrug analogues are attractive alternatives to teixobactin as antibiotic drug candidates that circumvent the gelation problem of teixobactin.

It will be understood that the embodiments presented in this section are exemplary in nature and are provided to support and extend the broader disclosure. These embodiments are not meant to confine or otherwise limit the scope of the claims being sought.

### Example 1: Synthesis of O-Acyl Isopeptide Teixobactin

The teixobactin O-acyl isopeptide prodrug analogues were synthesized by Fmoc-based solid-phase peptide synthesis (SPPS) using the commercially available Boc-Ser(Fmoc-Ile)-OH O-acyl isodipeptide building block in place of Ile₂ and Ser₃, Ile₆ and Ser₇, or both Ile₂ and Ser₃ and Ile₆ and Ser₇. This approach was used to synthesize prodrugs of the teixobactin analogues, Lys₁₀-teixobactin, Arg₁₀-teixobactin, and Leu₁₀-teixobactin. The Arg₁₀ analogue replaces the cyclic guanidinium group of allo-enduracididine (allo-End) with an acyclic guanidinium group and exhibits good antibiotic activity. The Lys₁₀ analogue also contains a positively charged residue and exhibits good antibiotic activity. The Leu₁₀ analogue is especially interesting, because it contains an uncharged residue and still exhibits good antibiotic activity. Although the lack of commercial sources of allo-enduracididine makes it more difficult to access the corresponding prodrugs of teixobactin, it is anticipated that the synthetic route described here should also allow the synthesis of teixobactin prodrugs having the allo-End₁₀.

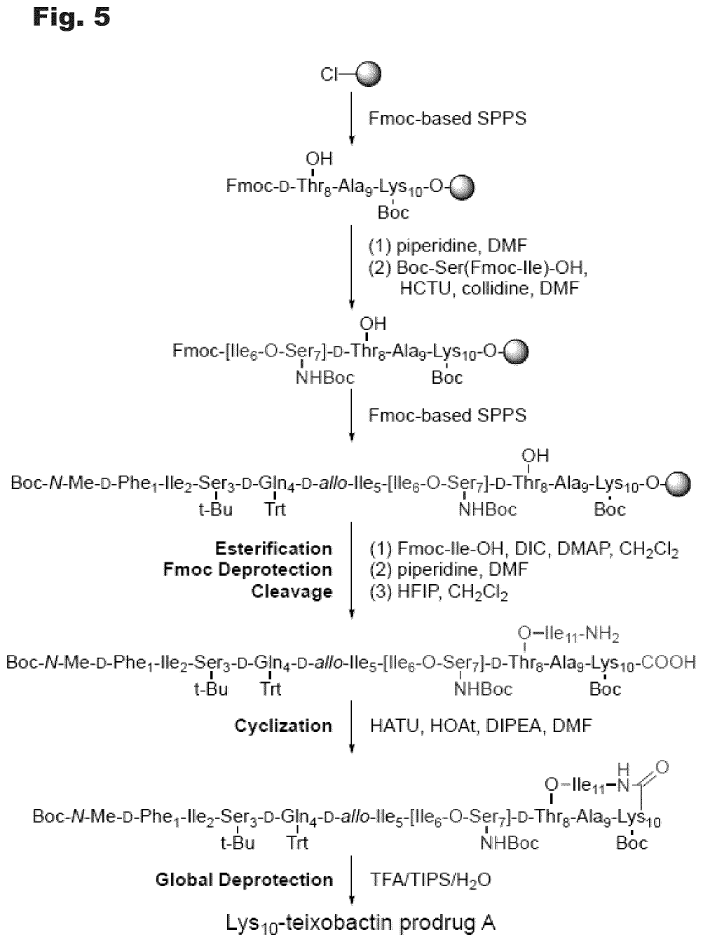

The synthesis of these O-acyl isopeptide prodrugs begins by attaching Fmoc-Lys(Boc)-OH, Fmoc-Arg(Pbf)-OH, or Fmoc-Leu-OH to 2-chlorotrityl chloride resin. Residues 9 through 1 are then introduced by standard Fmoc-based SPPS using HCTU as the coupling reagent. Boc-Ser(Fmoc-Ile)-OH is coupled in place of the desired lie and Ser residues to provide an ester linkage in place of an amide linkage. Ile₁₁ is then introduced through an esterification using DIC and DMAP. Fmoc deprotection followed by cleavage of the peptide from resin with 20% hexafluoroisopropanol (HFIP) in CH₂Cl₂ affords the selectively deprotected uncyclized peptide. Solution-phase macrolactamization with HATU and HOAt, followed by global deprotection with trifluoroacetic acid (TFA) and RP-HPLC purification affords the desired O-acyl isopeptide prodrug analogue of teixobactin as the trifluoroacetate salt. FIG. 5 illustrates this route with the synthesis of Lys₁₀-teixobactin prodrug A. Synthesis on a 0.1 mmol scale typically yields ca. 10 mg of the O-acyl isopeptide prodrug.

The trifluoroacetate salts of the O-acyl isopeptide teixobactin analogues are more soluble and easier to handle than the corresponding teixobactin analogues. When working with active teixobactin analogues, 30-40% aqueous acetonitrile is needed to dissolve the compounds for preparative HPLC injection, while only 10-20% acetonitrile is required for the O-acyl isopeptide teixobactin prodrug analogues. The introduction of the O-acyl isopeptide linkage thus improves the solubility and handling of the prodrugs by reducing their propensity to aggregate in aqueous solutions.

### Example 2: Conversion of O-Acyl Isopeptide Linkages

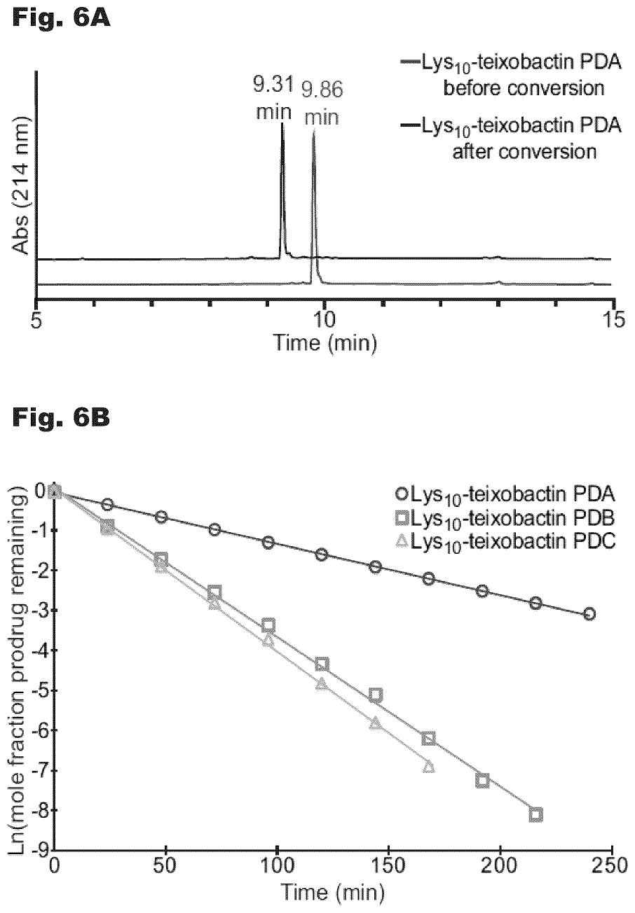

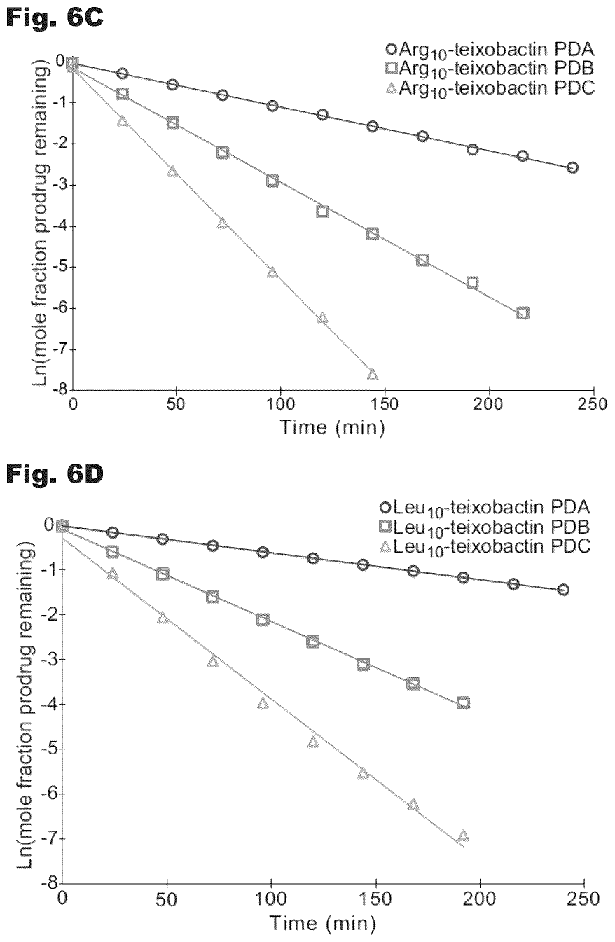

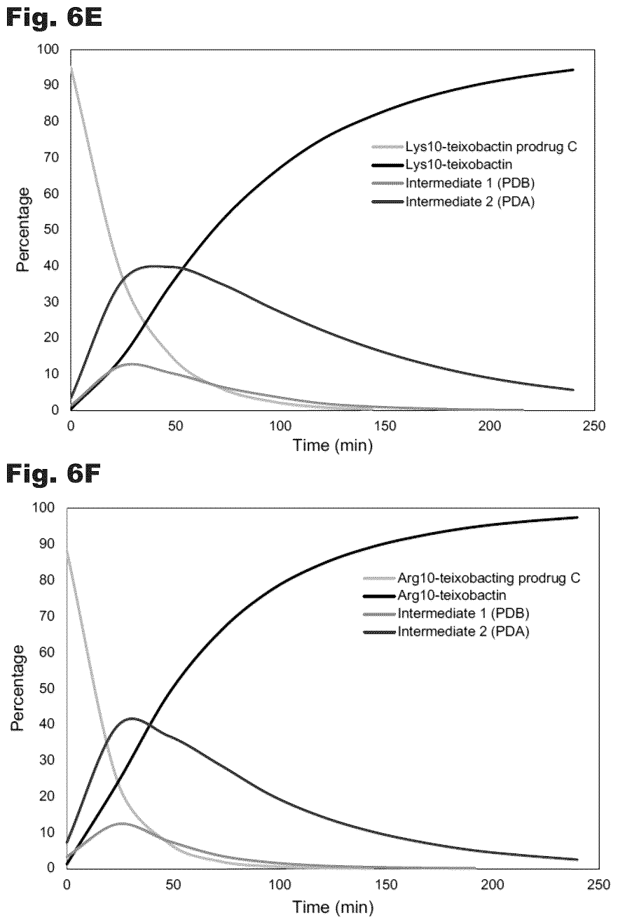

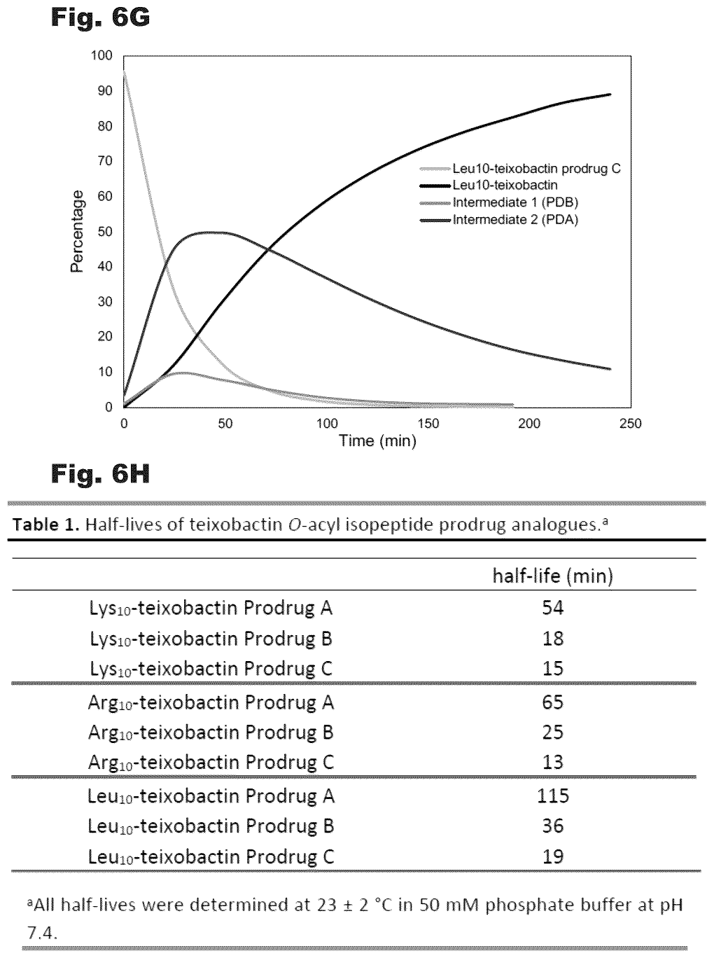

Each of the teixobactin prodrugs undergoes clean conversion to the corresponding teixobactin analogue at physiological pH. When each of the A and B series of prodrugs was incubated in 50 mM phosphate buffer at pH 7.4 and the conversion reaction was monitored by HPLC, a new peak appeared in the HPLC trace corresponding to the teixobactin analogue. FIG. 6A illustrates the clean conversion of Lys₁₀-teixobactin prodrug A to Lys₁₀-teixobactin. No intermediates were observed, and the conversion was complete within 12-24 h at 23±2° C. When each of the C series of prodrugs was incubated in 50 mM phosphate buffer under similar conditions, two intermediates were observed, and conversion to the corresponding teixobactin analogue was complete within 12-24 h. The intermediates correspond to the A and B series prodrugs, which form upon isomerization of the Ile₂-Ser₃ and Ile₆-Ser₇ isopeptide linkages.

The conversion of the A and B series of prodrugs to the corresponding teixobactin analogues shows clean first-order kinetics for the disappearance of the prodrugs and the appearance of the corresponding teixobactin analogues. FIG. 6B illustrates the conversion kinetics for the disappearance of Lys₁₀-teixobactin prodrugs A, B, and C. The conversion of the C series of prodrugs also shows clean first-order kinetics for the disappearance of the prodrugs (FIGS. 6B, 6C, and 6D). The A- and B-series prodrugs form as intermediates during conversion of the C series and then undergo subsequent conversion to the corresponding teixobactin analogues (FIGS. 6E, 6F and 6G). The A-series prodrugs exhibited half-lives of 54-115 minutes in phosphate buffer at 23±2° C., while the B- and C-series prodrugs exhibited half-lives of 13-36 minutes (FIG. 6H). The shorter half-lives of the B- and C-series prodrugs may reflect effects of the N-terminal methylammonium group on the pKₐ of the proximal Ser₃ ammonium group in the B- and C-series O-acyl isopeptides.

### Example 3: Antibiotic Activity of O-Acyl Isopeptide Teixobactin

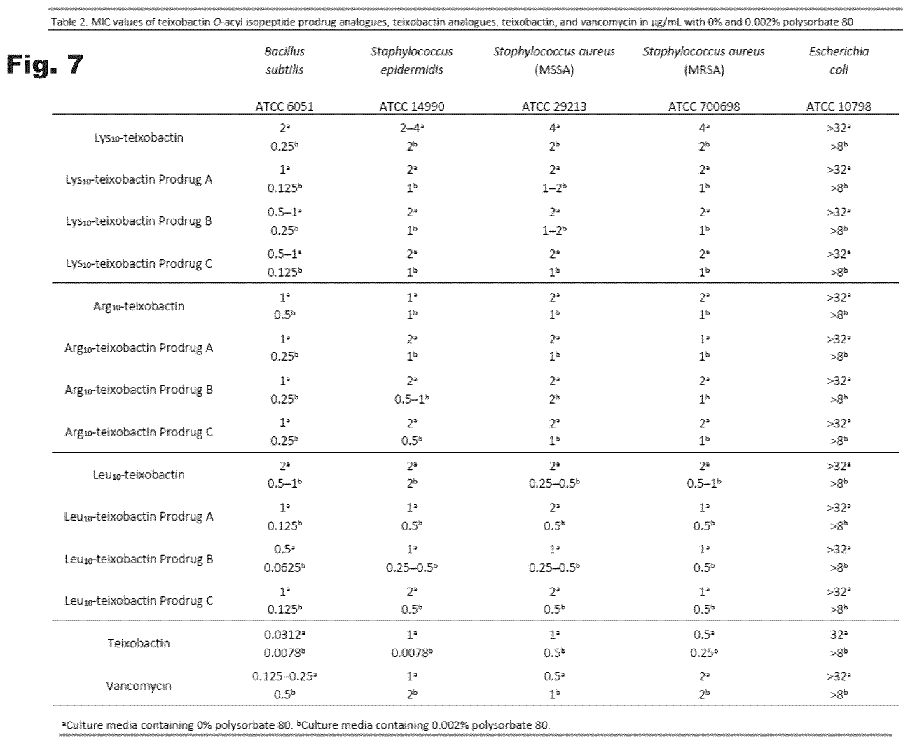

The O-acyl isopeptide prodrugs exhibit comparable or slightly improved antibiotic activity compared to the corresponding teixobactin analogues (FIG. 7). The antibiotic activity of the teixobactin O-acyl isopeptide prodrugs was evaluated using minimum inhibitory concentration (MIC) assays with four Gram-positive bacteria and compared the MIC values to those of the parent teixobactin analogues. Methicillin-susceptible and methicillin-resistant S. aureus were used as representative pathogens and B. subtilis and S. epidermidis as additional Gram-positive bacteria, and E. coli was used as a Gram-negative control. The activities of the teixobactin analogues and prodrugs were compared to those of teixobactin and vancomycin.

The Lys₁₀-teixobactin prodrugs showed slightly improved activity compared to Lys₁₀-teixobactin. Thus, the Lys₁₀-teixobactin prodrugs exhibited MICs of 0.5-2 μg/mL, while Lys₁₀-teixobactin exhibited MICs of 2-4 μg/mL. The Arg₁₀-teixobactin prodrugs showed comparable antibiotic activity to Arg₁₀-teixobactin, with MICs of 1-2 μg/mL. The Leu₁₀-teixobactin prodrugs showed equal or slightly improved antibiotic activity compared to Leu₁₀-teixobactin, with MICs of 0.5-2 μg/mL.

In the original report on teixobactin, the authors performed MIC assays in the presence of 0.002% polysorbate 80, with the rationale that the polysorbate 80 prevented teixobactin from sticking to plastic surfaces (see L. L. Ling, et al, Nature, 2015, 517, 455-459, the disclosure of which is incorporated herein by reference). When the MIC assays were performed with the Lys₁₀-, Arg₁₀-, and Leu₁₀-teixobactin analogues and the corresponding prodrugs A, B, and C in the presence of polysorbate 80, enhanced antibiotic activity was observed. Lys₁₀-teixobactin exhibited MICs of 0.25-2 μg/mL, and the Lys₁₀-teixobactin prodrugs exhibited MICs of 0.125-2 μg/mL. Arg₁₀-teixobactin and the Arg₁₀-teixobactin prodrugs exhibited MICs of 0.25-2 μg/mL. Leu₁₀-teixobactin exhibited MICs of 0.25-1 μg/mL, and the Leu₁₀-teixobactin prodrugs exhibited MICs of 0.0625-0.5 μg/mL.

Although the prodrugs themselves are not expected to exhibit antibiotic activity, conversion under the 37° C. assay conditions should be rapid enough—minutes to tens of minutes—to prevent the bacteria from propagating. The greater activity observed for some of the prodrugs may reflect higher effective drug concentrations resulting from complete dispersion of the prodrugs within the media. In the presence of 0.002% polysorbate 80, the Leu₁₀-teixobactin prodrugs (MIC 0.0625-0.5 μg/mL) are somewhat more active than vancomycin (MIC 0.125-2 μg/mL), although somewhat less active than teixobactin itself (MIC 0.0078-1 μg/mL).

### Example 4: Gelation Assay of O-Acyl Isopeptide Teixobactin

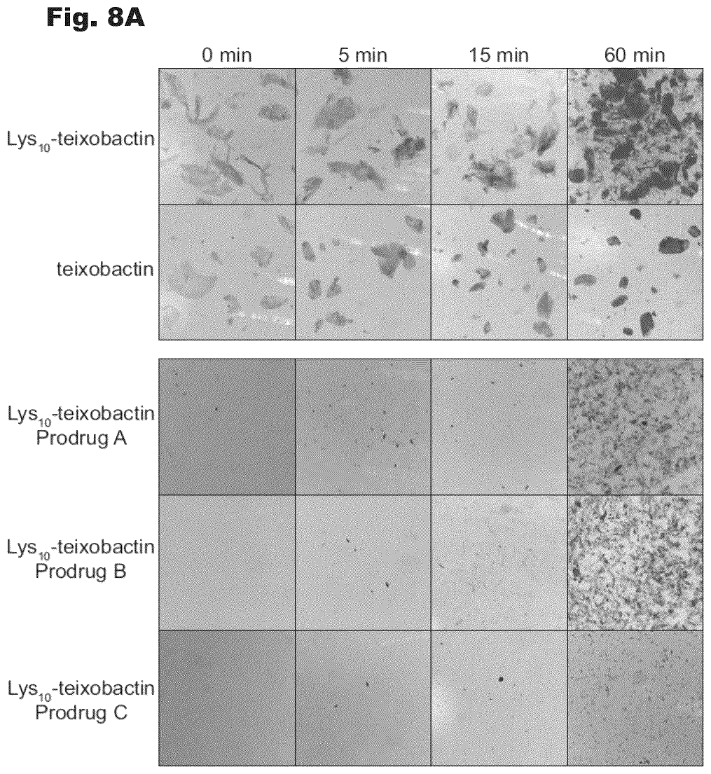

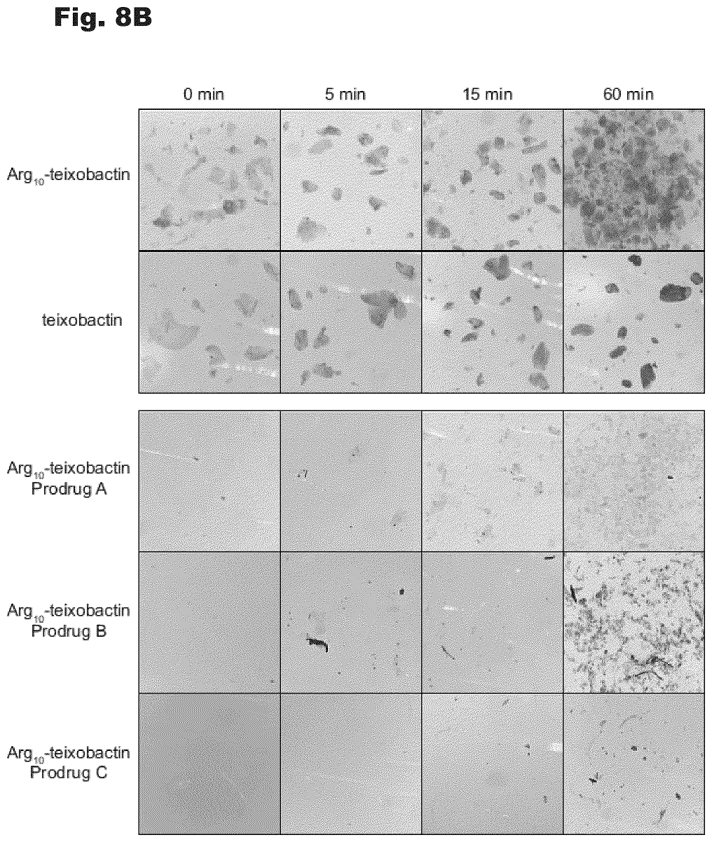

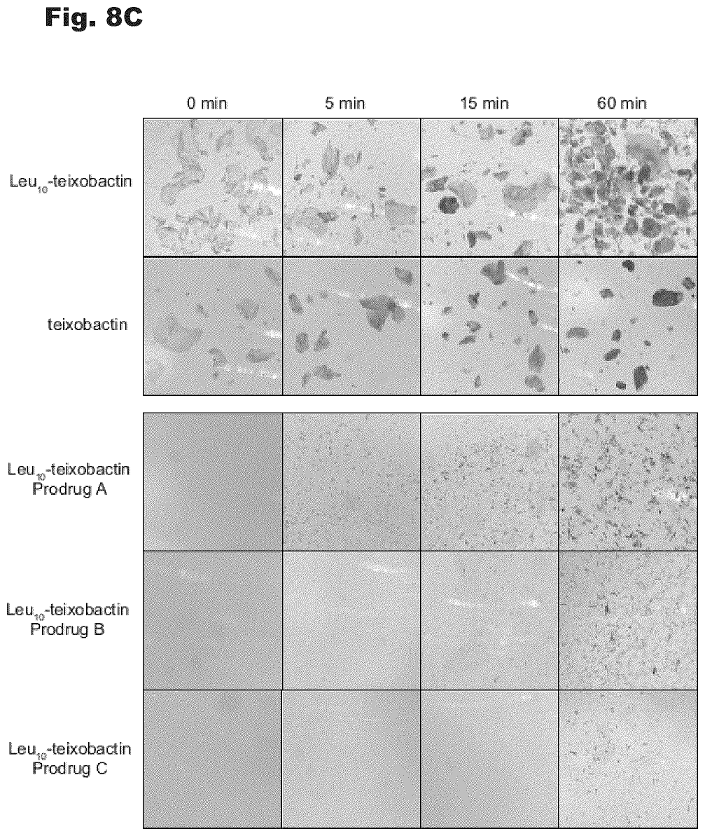

The O-acyl isopeptide prodrug analogues of teixobactin exhibit delayed gel formation at physiological pH. Gel formation of the teixobactin analogues was compared to that of the O-acyl isopeptide prodrugs in a qualitative gelation assay. In this experiment, a 10 mg/mL stock solution of the peptide trifluoroacetate salt in DMSO is added to 1×PBS buffer at pH 7.4 and gel formation is observed over time. Crystal violet is added to the PBS buffer to facilitate the visualization of the gels by providing contrast. When Lys₁₀-teixobactin is added to PBS, large gelatinous aggregates form immediately (FIG. 8A). Similar behavior is also observed for teixobactin (as the hydrochloride salt). In contrast, when the Lys₁₀-teixobactin prodrugs are added, no immediate gel formation occurs. After 5 mins, a few small gelatinous aggregates become visible, with Lys₁₀-teixobactin prodrug C showing the least amount of gel formation. After 15 mins, the number of aggregates increases but the size of the aggregates does not. By 60 mins, gel formation increases significantly, especially for Lys₁₀-teixobactin prodrugs A and B, which also begin to form gelatinous aggregates of larger size. The Arg₁₀-teixobactin prodrugs and Leu₁₀-teixobactin prodrugs show similar behavior, not immediately forming gels when added to PBS, and then forming aggregates over 60 minutes (FIGS. 8B and 8C). In contrast, Arg₁₀-teixobactin and Leu₁₀-teixobactin form gels immediately upon addition to PBS.

The gelation assays of the O-acyl isopeptide prodrugs demonstrate that these compounds do not gelate immediately upon exposure to buffer, unlike teixobactin and active teixobactin analogues. Thus, the prodrugs remain in solution and can be thoroughly dispersed in PBS. As the prodrugs gradually convert, they form aggregates that are smaller and more dispersed than those formed by teixobactin and the teixobactin analogues. The greater solubility of the prodrugs should impart better pharmacological properties than the parent analogues and may thus make them superior drug candidates.

### Example 5: Hemolytic and Cytotoxicity Assays of O-Acyl Isopeptide Teixobactin

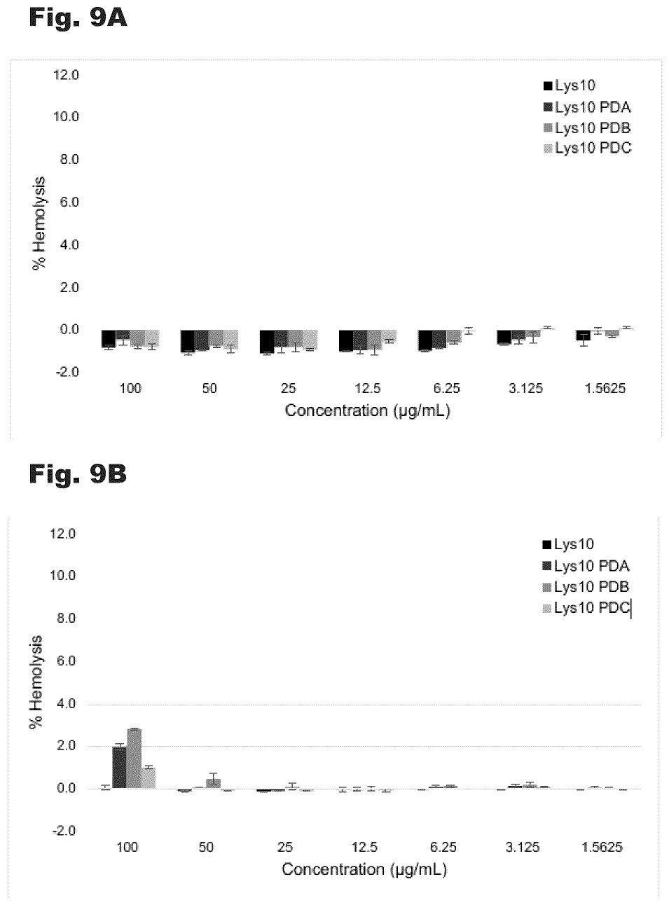

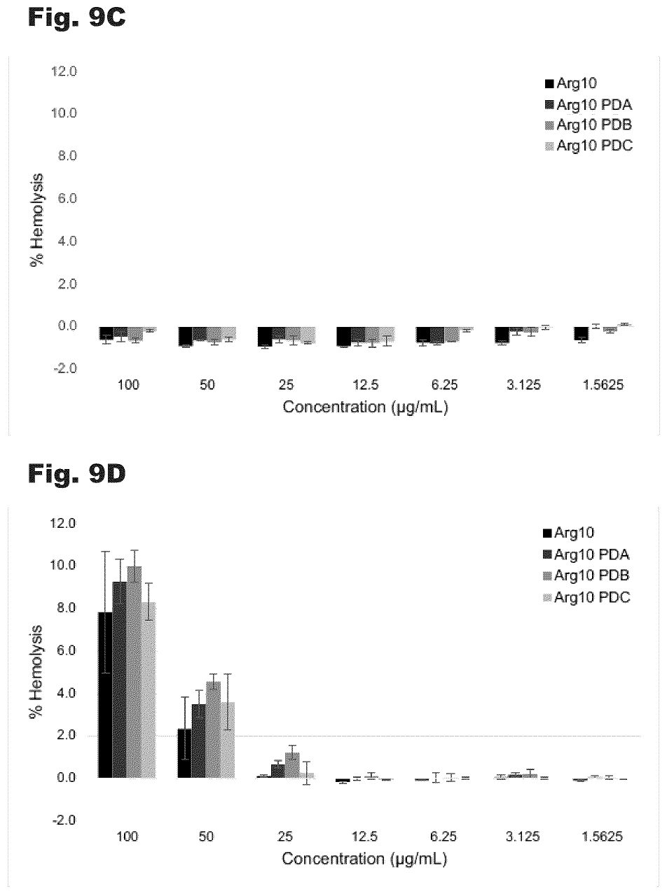

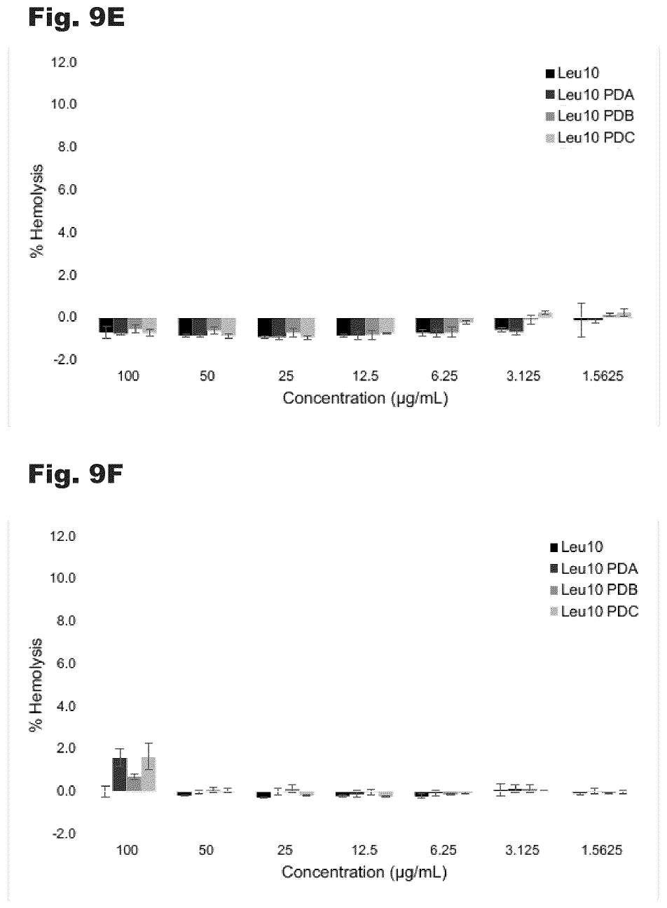

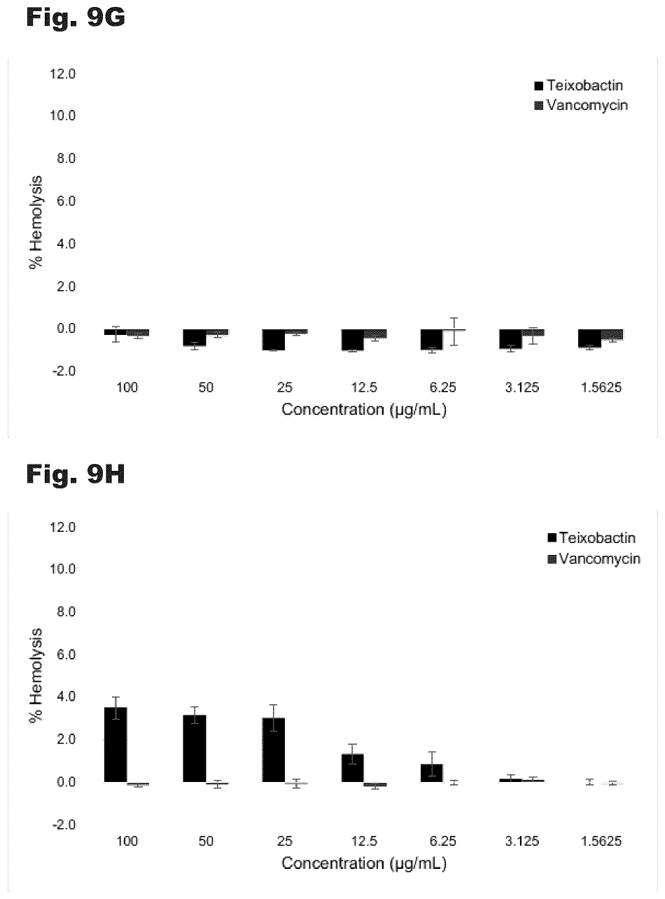

The hemolytic activity of the O-acyl isopeptide prodrugs and the corresponding teixobactin analogues were evaluated with human red blood cells (FIGS. 9A to 9H). Triton X-100 and melittin were used as positive controls and vancomycin and water (vehicle) were used as negative controls in the hemolysis assays. The teixobactin analogues and corresponding O-acyl isopeptide prodrugs exhibited no hemolytic activity at concentrations up to 100 μg/mL in the absence of polysorbate 80. In the presence of 0.002% polysorbate 80, the Arg₁₀-teixobactin analogue and corresponding O-acyl isopeptide prodrugs exhibited modest hemolytic activity, with 7-10% hemolytic activity occurring at 100 μg/mL. In contrast, 0.002% polysorbate 80 had no effect on the hemolytic activity of Lys₁₀-teixobactin and Leu₁₀-teixobactin and little effect on the corresponding O-acyl isopeptide prodrug analogues. When the hemolysis assays were performed with teixobactin, no hemolysis was observed up to 100 μg/mL without polysorbate 80 and modest hemolysis (4%) at 100 μg/mL with 0.002% polysorbate 80. No hemolysis was observed with vancomycin at concentrations up to 100 μg/mL, and 26-30% hemolysis with 1.25 μg/mL melittin was observed with and without 0.002% polysorbate 80. Collectively, these studies suggest the O-acyl isopeptide prodrug analogues should be suitable for intravenous administration at concentrations well above the MIC values.

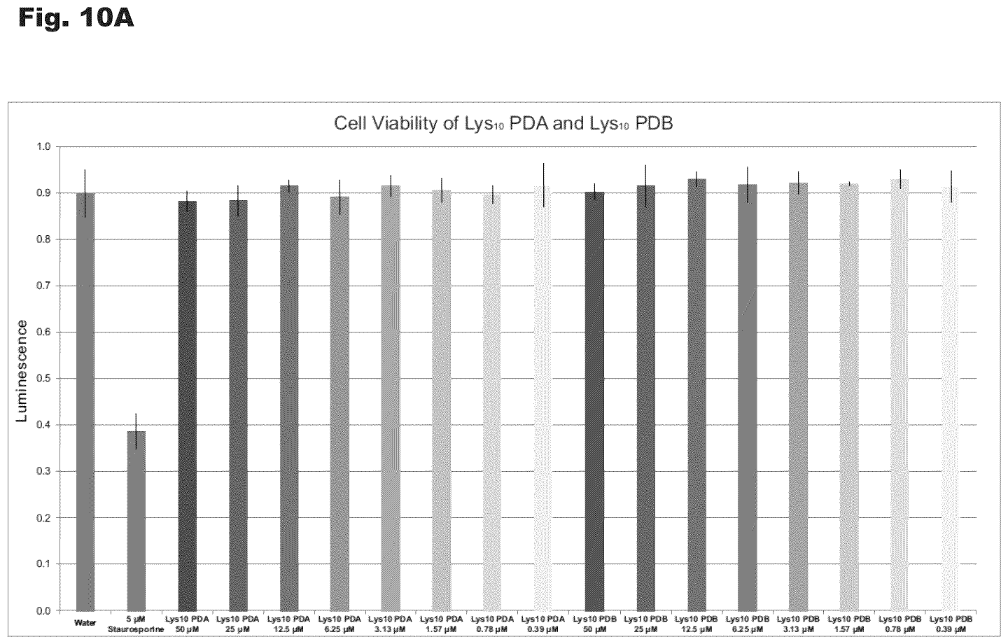

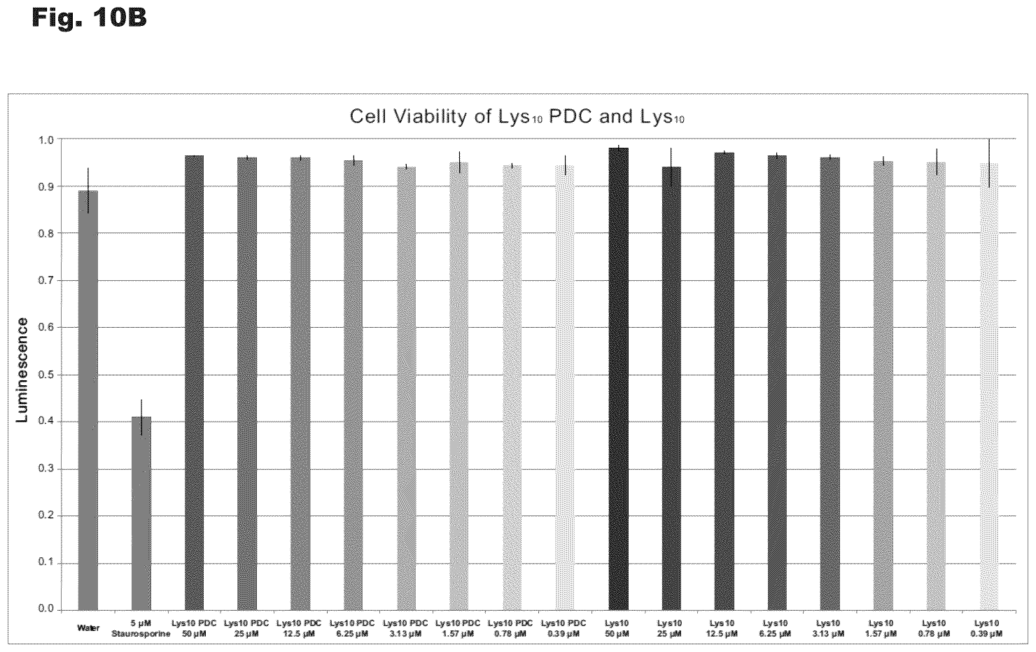

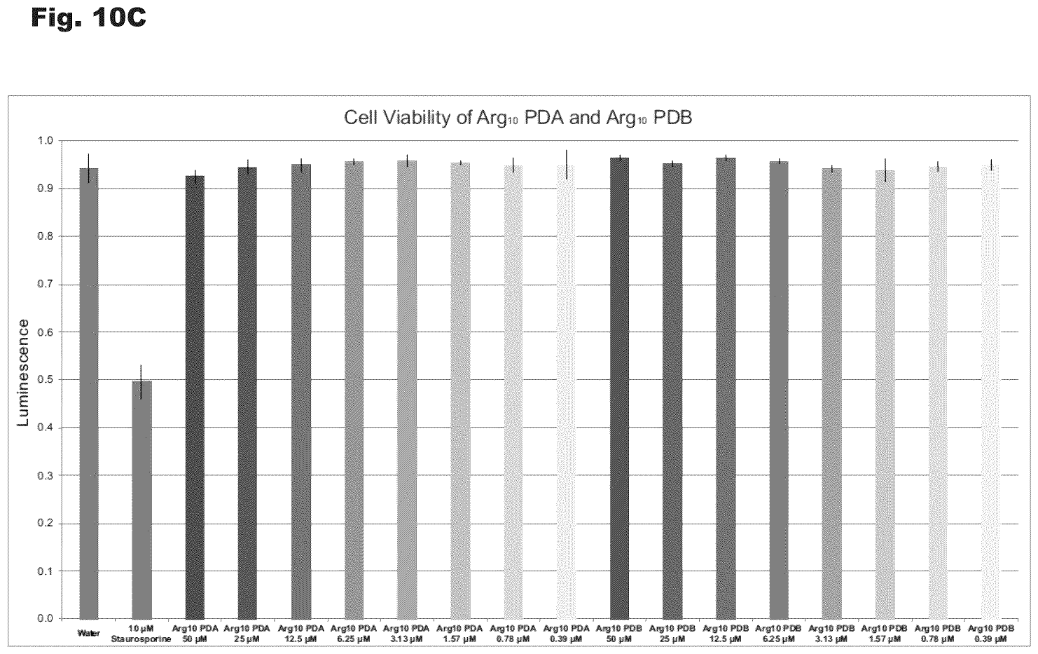

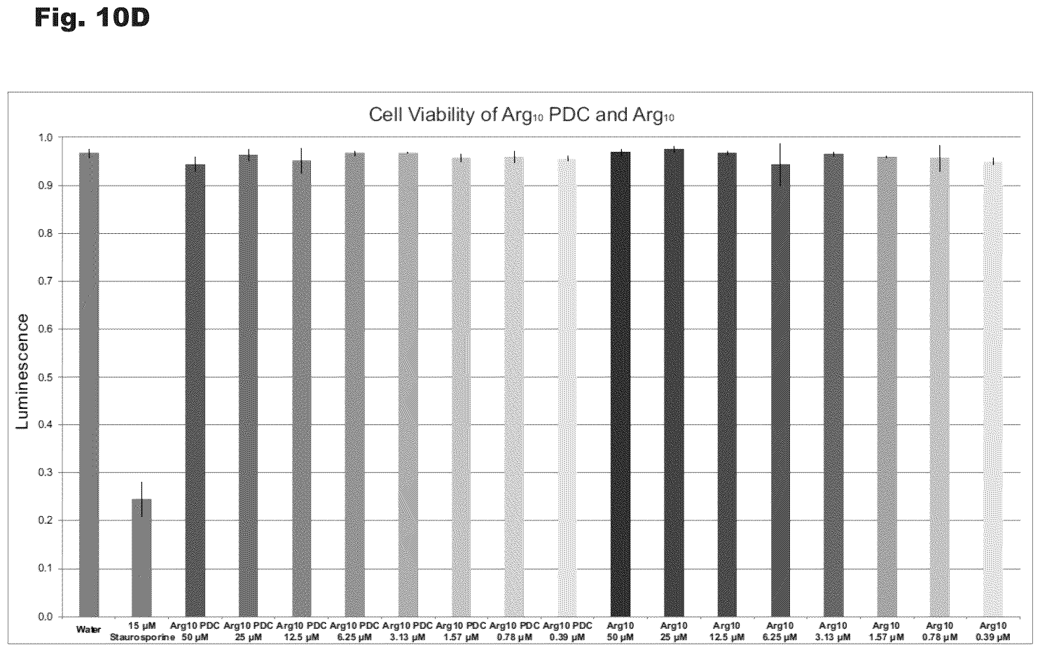

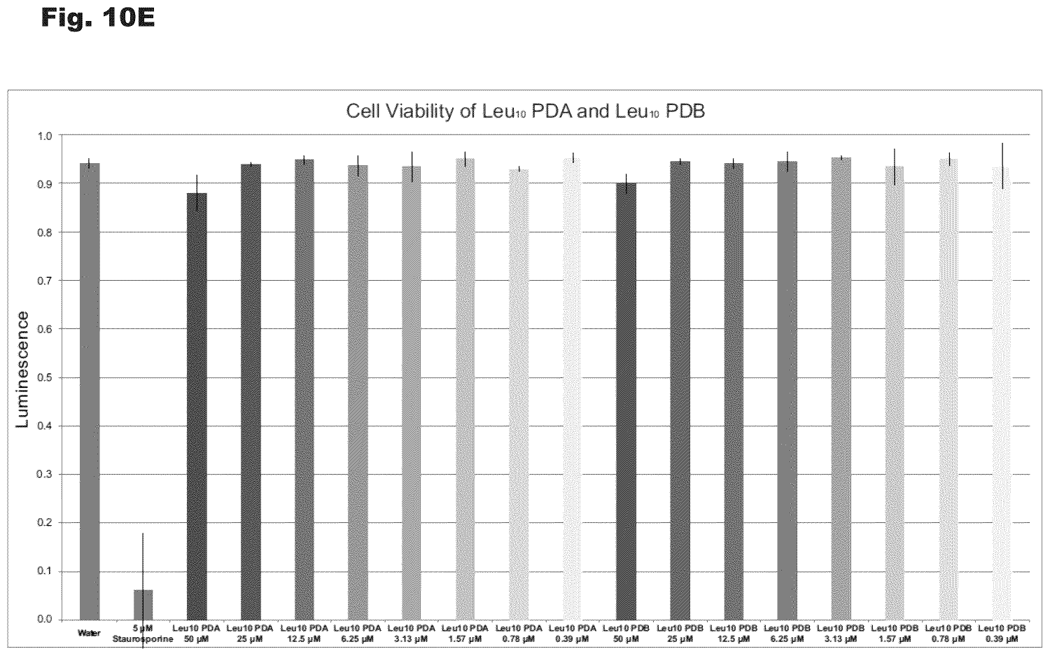

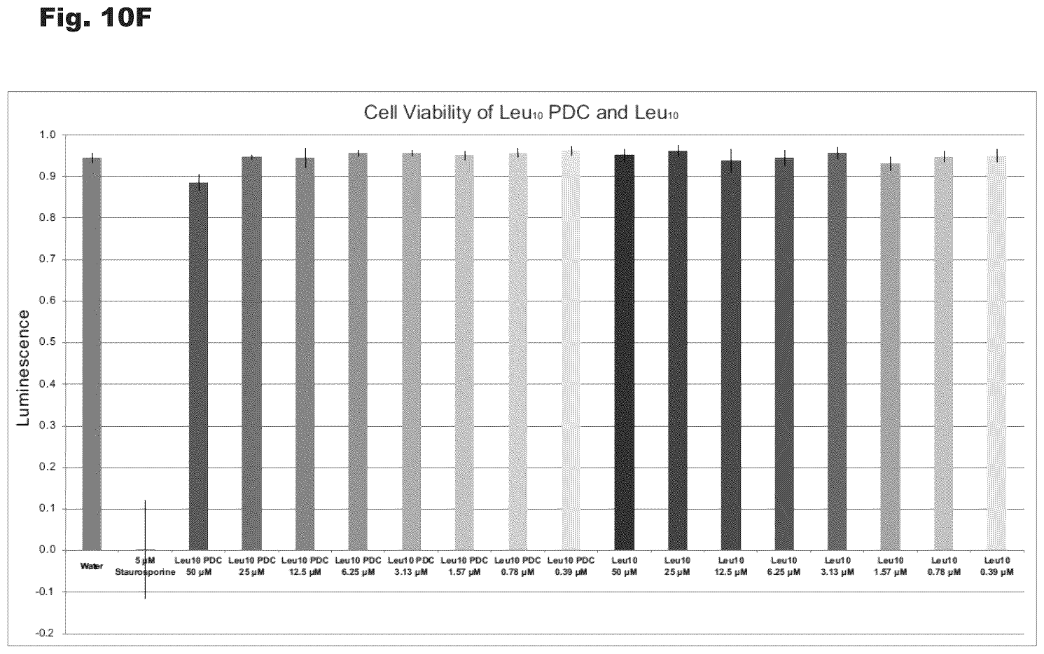

To further assess the potential of the teixobactin O-acyl isopeptide prodrug analogues as potential drugs, cytotoxicity assays were performed (FIGS. 10A to 10F). The cytotoxicity of the teixobactin O-acyl isopeptide prodrugs and the corresponding teixobactin analogues was evaluated on HeLa cells using a Promega Cytotox-Glo assay. In these experiments, the Lys₁₀- and Arg₁₀-teixobactin O-acyl isopeptide prodrugs exhibited no cytotoxicity at concentrations up to 50 μM (72-79 μg/mL). The Leu₁₀-teixobactin O-acyl isopeptide prodrugs exhibited no cytotoxicity at concentrations up to 25 μM (33-36 μg/mL) and slight cytotoxicity at 50 μM (66-71 μg/mL). These studies further suggest that the O-acyl isopeptide prodrug analogues should be suitable for intravenous administration at concentrations well above the MIC values.

### Materials and Methods Supporting the Various Examples

### Materials

Amino acids, coupling agents, 2-chlorotrityl chloride resin, DIC, and triisopropylsilane were purchased from Chem-Impex. Boc-Ser(Fmoc-Ile)-OH was purchased from AAPPTec. Vancomycin (hydrochloride salt) was purchased from Sigma-Aldrich. Teixobactin (hydrochloride salt) was provided as the by NovoBiotic Pharmaceuticals. DMF (amine-free), DIPEA, 2,4,6-collidine, and piperidine were purchased from Alfa-Aesar. DMAP and polysorbate 80 were purchased from Acros Organics. HPLC-grade acetonitrile, and dichloromethane were purchased from Fisher Scientific. TFA and hexafluoroisopropanol were purchased from Oakwood Chemical. Reagent-grade solvents, chemicals, amino acids, and resin were used as received, with the exception of dichloromethane, which was dried through an alumina column under argon, and DMF, which was dried through an alumina column and an amine scavenger resin column under argon.

### Methods for Synthesis, Purification, and Analysis of Peptides

Solid-phase peptide synthesis was carried out manually in a solid phase reaction vessel. Analytical reverse-phase HPLC was performed on an Agilent 1260 instrument equipped with an Aeris PEPTIDE 2.6 μm XB-C18 column (Phenomonex). Preparative reverse-phase HPLC was performed on a Rainin Dynamax instrument equipped with a Zorbax SB-C18 column (Agilent) for all teixobactin analogues. All teixobactin prodrug analogues were first purified on a Biotage® Isolera™ One system equipped with a Biotage® Sfär Bio C18—Duo 300 A 20 μm column, before repurifying on the Rainin Dynamax instrument. UV detection (214 nm) was used for analytical and preparative HPLC. HPLC grade acetonitrile and 18 MO deionized water, each containing 0.1% trifluoroacetic acid, were used for analytical and preparative reverse-phase HPLC. Matrix-assisted laser desorption/ionization time-of-flight (MALDI-TOF) mass spectrometry was performed on an AB SCIEX TOF/TOF 5800 system and α-cyano-4-hydroxycinnamic acid was used as the sample matrix. All peptides were prepared and used as the trifluoroacetate salts and were assumed to have one trifluoroacetate ion per ammonium group present in each peptide.

Synthesis of Teixobactin O-Acyl Isopeptide Prodrug Analogues and their Corresponding Teixobactin Analogues

Lys₁₀-teixobactin, Arg₁₀-teixobactin, Leu₁₀-teixobactin, and all O-acyl isopeptide prodrug analogues were prepared as the trifluoroacetate salts by solid-phase peptide synthesis followed by solution phase cyclization. HATU and HOAt were used in the solution-phase cyclization step the synthesis of these peptides. For all O-acyl isopeptide prodrug analogues, Boc-Ser(Fmoc-Ile)-OH was coupled in place of the desired lie and Ser residues. Syntheses on a 0.1-0.2 mmol scale afforded 5-39 mg (1.6-21%) of Lys₁₀-teixobactin, Arg₁₀-teixobactin, Leu₁₀-teixobactin and the O-acyl isopeptide prodrug analogues.

### Representative Synthesis of Lys₁₀-Teixobactin Prodrug A

Resin Loading. 2-chlorotrityl chloride resin (300 mg, 1.6 mmol/g) was added to a 10-mL Bio-Rad Poly-Prep chromatography column. The resin was suspended in dry DCM (8 mL) and allowed to swell for 10 mins. The DCM was drained and a solution of Fmoc-Lys(Boc)-OH (150 mg, 0.32 mmol, 1.8 equiv) and 2,4,6-collidine (300 μL) in dry DCM (7 mL) was added. The suspension was gently agitated for 5 h. The solution was drained, and the resin was washed with dry DCM (3×). After washing, a solution of DCM/MeOH/DIPEA (17:2:1, 8 mL) was added to the resin and agitated for 1 h to cap any unreacted 2-chlorotrityl chloride sites. The solution was drained, and the resin was washed with DCM (3×) and dried with a flow of nitrogen. The resin loading was determined to be 0.18 mmol (0.60 mmol/g, 57% loading) based on UV analysis (290 nm) of the Fmoc cleavage product.

Solid-phase amino acid couplings. The loaded resin was suspended in dry DMF and transferred to a solid-phase peptide synthesis reaction vessel for manual peptide synthesis. Fmoc-Ala-OH, Fmoc-d-Thr-OH, Boc-Ser(Fmoc-Ile)-OH, Fmoc-d-allo-Ile-OH, Fmoc-d-Gln(Trt)-OH, Fmoc-Ser(t-Bu)-OH, Fmoc-Ile-OH, and Boc-N-methyl-d-Phe-OH were coupled through the following cycles: (1) Fmoc deprotection with 20% (v/v) piperidine in dry DMF (5 mL) for 5 min (2×), (2) resin washing with dry DMF (7×), (3) coupling of amino acid (0.72 mmol, 4.0 equiv) with HCTU (0.72 mmol, 4.0 equiv) in 20% (v/v) collidine in dry DMF (5 mL) for 30 min, and (4) resin washing with dry DMF (7×). After completing the linear synthesis, the resin was transferred to a 10-mL Bio-Rad Poly-Prep chromatography column and washed with dry DMF (3×) and DCM (3×).

Esterification. In a test tube, Fmoc-Ile-OH (630 mg, 1.8 mmol, 9.9 equiv) and diisopropylcarbodiimide (280 μL, 1.8 mmol, 10 equiv) were dissolved in dry DCM (5 mL). The solution was filtered through a 0.20-μm nylon filter into a test tube containing 4-dimethylaminopyridine (21.8 mg, 0.18 mmol, 1.00 equiv). The filtrate was transferred to the resin and gently agitated for 1 h. The solution was drained, and the resin washed with dry DCM (3×) and DMF (3×).

Fmoc deprotection of Ile₁₁. The Fmoc protecting group on Ile₁₁ was removed with a solution of 20% (v/v) piperidine in DMF (5 mL) for 15 mins. The solution was drained, and the resin was washed with dry DMF (3×) and DCM (3×).

Cleavage of the linear peptide from the resin. The linear peptide was cleaved from resin by subjecting the resin to a cleavage solution of 20% (v/v) HFIP in dry DCM (7.5 mL) and agitating for 1 h. The filtrate was collected in a 250-mL round-bottom flask. The HFIP treatment was repeated for 30 mins and the filtrate was added to the first in the round-bottom flask. The resin was washed with dry DCM (3×). The combined filtrates and DCM washes were concentrated under reduced pressure to afford a colorless oil.

Solution-phase cyclization. The oil was dissolved in DMF (125 mL) in the same 250 round-bottom flask as the previous step. HATU (410 mg, 1.1 mmol, 6.0 equiv) and HOAt (150 mg, 1.1 mmol, 6.1 equiv) were added to the solution. The reaction mixture was then stirred under nitrogen for 10 mins. DIPEA (100 μL, 0.6 mmol, 3.2 equiv) was added dropwise to the solution and the mixture was stirred under nitrogen at room temperature for 12 h. The reaction mixture was concentrated under reduced pressure to afford the cyclized peptide as a yellow solid. The solid was placed under vacuum (560 mTorr) to remove any residual solvents.

Global Deprotection and Ether Precipitation. The crude protected peptide was dissolved in a mixture of TFA/TIPS/H₂O (90:5:5, 10 mL), and the solution was stirred for 1.5 h. The deprotection mixture was transferred to two 50-mL conical tubes, each containing 35 mL ice-cold diethyl ether, with a precipitate forming immediately. The 50-mL conical tubes were centrifuged (2500×g) for 10 min to pellet the crude peptide. The diethyl ether supernatant was decanted into a 125-mL Erlenmeyer flask. This process was repeated 2×, adding additional ice-cold ether followed by centrifugation and decantation. The pellet was then dried under nitrogen.

Purifications. The dried peptide pellet was dissolved in 10% (v/v) MeCN in H₂O (10 mL) and purified on a Biotage® Isolera™ One system equipped with a Biotage® Sfär Bio C18—Duo 300 A 20 μm column using a H₂O/MeCN (10%-55%) gradient. The fractions were analyzed by MALDI-TOF and analytical HPLC. Fractions containing the desired peptide were combined and lyophilized for repurification. The lyophilized material from the first purification were dissolved in 20% (v/v) MeCN in H₂O (4 mL) and purified by reverse-phase HPLC with H₂O/MeCN (gradient elution of 20-40% with 0.1% TFA over 120 min) on a C18 column. Fractions were analyzed by MALDI-TOF and analytical HPLC. The pure fractions were combined and lyophilized to give 39 mg (14% yield based on resin loading) of Lys₁₀-teixobactin prodrug A trifluoroacetate (TFA) salt as a white powder.

### Conversion Kinetics Studies of Teixobactin O-Acyl Isopeptide Prodrug Analogues

A 1 mL analytical HPLC vial was charged with 300 μL of 50 mM phosphate buffer followed by 300 μL of a 1 mg/mL stock solution of the peptide in H₂O. An aliquot was then immediately injected onto an Agilent 1260 instrument equipped with an Aeris PEPTIDE 2.6 μm XB-C18 column (Phenomonex). Additional aliquots were injected every 24 minutes for 4 h. Each sample was run on a gradient of 5-67% acetonitrile over 15 min, with monitoring of absorbance at 214 nm. The temperature in the HPLC sample chamber was recorded. Conversion kinetics of all reactions were run at 23-25±1° C. Peaks corresponding to the prodrug analogue, intermediates (prodrugs C only), and the teixobactin analogue product were integrated using the Agilent software, and the relative areas were recorded for kinetic analysis.

MIC Assays of Teixobactin O-Acyl Isopeptide Prodrug Analogues and their Corresponding Teixobactin Analogues

Bacillus subtilis (ATCC 6051), Staphylococcus epidermidis (ATCC 14990), Staphylococcus aureus (ATCC 29213), and Escherichia coli(ATCC 10798) were cultured from glycerol stocks in Mueller-Hinton broth overnight in a shaking incubator at 37° C. Staphylococcus aureus (ATCC 700698) was cultured from a glycerol stock in brain heart infusion broth overnight in a shaking incubator at 37° C. An aliquot of a 1 mg/mL antibiotic stock solution in DMSO was diluted with appropriate culture media to make a 64 μg/mL solution. A 200-μL aliquot of the 64 μg/mL solution was transferred to a sterile, untreated 96-well plate. Two-fold serial dilutions were made with media across a 96-well plate to achieve a final volume of 100 μL in each well. These solutions had the following concentrations: 64, 32, 16, 8, 4, 2, 1, 0.5, 0.25, 0.125, and 0.0625 μg/mL. The overnight cultures of each bacterium were diluted with Mueller-Hinton broth to an OD₆₀₀ of 0.075 as measured for 200 μL in a 96-well plate. The diluted mixture was further diluted to a 1×10⁶ CFU/mL with Mueller-Hinton media. A 100-μL aliquot of the 1×10⁶ CFU/mL bacterial solution was added to each well in the 96-well plates, resulting in final bacteria concentrations of 5×10⁵ CFU/mL in each well. As 100-μL of bacteria were added to each well, the teixobactin analogues and teixobactin prodrug analogues were also diluted to the following concentrations: 32, 16, 8, 4, 2, 1, 0.5, 0.25, 0.125, 0.0625, and 0.03125 μg/mL. The plate was covered with a lid and incubated at 37° C. for 16 h. The OD₆₀₀ were measured using a 96-well UV/vis plate reader (MultiSkan GO, Thermo Scientific). The MIC values were taken as the lowest concentration that had no bacteria growth. Each MIC assay was run in quadruplicate (technical replicates). MIC assays were performed in test media without polysorbate 80 or containing 0.002% polysorbate 80. For MIC assays performed with 0.002% polysorbate 80, the antibiotic stock solution was diluted with appropriate culture media to make a 16 μg/mL solution. Several of the MIC assays were repeated to ensure reproducibility.

Gel Formation Studies of Teixobactin O-Acyl Isopeptide Prodrug Analogues and their Corresponding Teixobactin Analogues

Gelation assays of the O-acyl isopeptide prodrug analogues of teixobactin were performed using procedures previously reported. See K. Chen, et al., Chem. Commun. 2017, 53, 11357-11359, the disclosure of which is herein incorporated by reference.

Hemolytic Assay of Teixobactin O-Acyl Isopeptide Prodrug Analogues and their Corresponding Teixobactin Analogues

Preparation of Phosphate-Buffered Saline (PBS) Buffers. A 10×PBS buffer was prepared by dissolving 8.9 g of Na₂HPO₄, 1.2 g KH₂PO₄, 40 g NaCl, and 1 g KCl in 500 mL of 18 MO deionized water. The solution was stirred until the buffer salts were completely dissolved. The pH of the 10×PBS buffer was adjusted to 7.4 using either 1 M HCl or 1 M NaOH and was subsequently sterile filtered. To create a 1×PBS buffer, the 10×PBS buffer was diluted 10-fold using 18 MO deionized water. Another 1×PBS buffer was made, supplemented with 0.002% polysorbate 80.

Preparation of human red blood cells. Whole human blood was stored in a 4° C. in K2 EDTA to prevent coagulation. On the day of cell treatment, the blood was centrifuged at 800×g for 5 min at 4° C. to isolate red blood cells (RBCs). The plasma layer was then removed and discarded. Approximately 3 mL of 150 mM NaCl solution was added to the RBCs and mixed gently by inversion. The RBCs were centrifuged at 800×g for 8 min at 4° C. and the supernatant was discarded. An additional wash with 150 mM NaCl was performed, centrifuged at 800×g for 8 min at 4° C. and the supernatant was discarded. 2 mL of whole RBCs were transferred to a 15-mL conical tube. Approximately 4 mL of 1×PBS was added to the RBCs and inverted gently to mix. The RBCs were centrifuged at 800×g for 8 min at 4° C. The supernatant was discarded, and the cells were washed 2-3 more times to ensure that the supernatant was visibly transparent and free of any color from pre-existing lysed RBCs. After the PBS washes, a 5% v/v RBC suspension was prepared by adding 500 μL of the RBCs to 9.5 mL of the desired 1×PBS.

Hemolytic assay procedure. Experiments were performed in triplicate (three technical replicates) in untreated V-bottom 96-well plates. An aliquot of a 1 mg/mL antibiotic stock solution in H₂O was diluted with the proper 1×PBS to make a 200 μg/mL solution. A 100-μL aliquot of the 200 μg/mL solution was transferred to a V-bottom 96-well plate. Two-fold serial dilutions were made with the desired 1×PBS down the V-bottom 96-well plate to achieve a final volume of 50 μL in each well. These solutions had the following concentrations: 200, 100, 50, 25, 12.5, 6.25, 3.125 μg/mL. The final row was used for controls, with each well receiving a 50-μL aliquot of the appropriate control. Four wells were used for a positive control with 4% Triton X-100 solution in 1×PBS. Four wells were used for a peptidic positive control with a 2.5 μM melittin solution in 1×PBS. Four wells were used for a vehicle control with 0.98×PBS (1×PBS diluted with 2% 18 MO deionized water). A 50-μL aliquot of the 5% RBC suspension was added to each well in the V-bottom 96-well plates. After addition of the RBCs to each well, the concentrations of the peptides were: 100, 50, 25, 12.5, 6.25, 3.125, and 1.5625 μg/mL, and the concentrations of the controls were 2% Triton X-100 and 1.25 μM melittin. The plates were sealed with a Axygen AxySeal Sealing Film and incubated at 37° C. for 1 h.

Hemolytic assay readout. A replica plate was prepared by adding a 50-μL aliquot of 1×PBS to all wells of a flat-bottomed 96-well plate. After the 1 h incubation period, the V-bottom 96-well plate was centrifuged at 1000×g for 10 min at 4° C. to pellet the RBCs. A 50-μL aliquot of the supernatant from each well was transferred to the replica plate. The transfer was performed quickly, but very carefully to not disturb the RBC pellet. [if any RBCs were disturbed, the V-bottom 96-well plate should be centrifuged again to re-pellet the RBCs.] The final volume of each well in the flat-bottom 96-well plate was 100 μL. The OD₅₄₀ of each well was measured using a 96-well UV/vis plate reader (MultiSkan GO, Thermo Scientific). The data were processed by comparing those values to the Triton X-100 controls and vehicle controls:

% hemolytic activity=[(A₅₄₀)_{compound}−(A₅₄₀)_{vehicle}]/[(A₅₄₀)ₜᵣᵢₜₒₙ−(A₅₄₀)_{vehicle}]×100

Cell Culture and Cytotoxicity Assays of Teixobactin O-Acyl Isopeptide Prodrug Analogues and their Corresponding Teixobactin Analogues

Cell culture. HeLa cell cultures were maintained in complete media of Eagle's Minimum Essential Medium (EMEM) supplemented with 10% heat-inactivated fetal bovine serum (FBS), 100 μg/mL penicillin, and 100 μg/mL streptomycin at pH 7.4 in a humidified 5% CO₂ atmosphere at 37° C. using a Fischer Scientific Forma Series 3 Water Jacketed CO₂ Incubator. All experiments were performed in triplicate in sterile half-area 96-well plates that were cell-culture treated.

Plating cells. HeLa cells were seeded at 2,500 cells per well in the inner 60 wells of half-area 96-well plates to a total volume of 50 μL using complete media. The outer wells of the plate were filled with 100 μL of EMEM without any cells. The plates were incubated in a 5% CO₂ atmosphere at 37° C. for 24 h after plating. Prior to treatment with peptide, the media was removed by pipet from the cells.

Treatment of cells with peptide. An aliquot of a 1 mg/mL antibiotic stock solution in H₂O was diluted with EMEM to make a 50 μM solution. A 100-μL aliquot of the 50 μM solution was transferred to the sterile, half-area 96-well plate. Two-fold serial dilutions were made with EMEM across a 96-well plate to achieve a final volume of 50 μL in each well. Each treatment was run in triplicate (technical replicates). An additional six wells were used as controls. Three wells received 50 μL of a 7% solution of 18 MO deionized water in EMEM (vehicle control) and the other three wells received 50 μL of either a 5, 10, or 15 μM staurosporine in EMEM solution (positive control). The plates were then incubated in a 5% CO₂ atmosphere at 37° C. for 48 h.

Cytotoxicity assay plate readout. The CytoTox-Glo Assay (CytoTox-Glo™ Cytotoxicity Assay, Promega) was performed according to manufacturer's instructions. Luminescence was measured using a microplate reader (GloMax® Discover System, Promega).

### DOCTRINE OF EQUIVALENTS

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

1. A peptide comprising:
a teixobactin peptide or a teixobactin analogue, or an enantiomer of the teixobactin peptide or the teixobactin analogue, having one or more O-acyl isopeptide linkages between two amino acids in place of an amide peptide bond.

2. The peptide of claim 1, wherein the one or more O-acyl isopeptide linkages is capable of chemically converting into amide peptide bonds at physiological conditions.

3. The peptide of claim 2, wherein chemical conversion of an O-acyl isopeptide linkage results in one of the two amino acids to have an alcohol side chain.

4. The peptide of claim 1, wherein the one or more O-acyl isopeptide linkages is between an amino acid with having a hydroxy group at the beta position and the immediately preceding amino acid.

5. The peptide of claim 1, wherein the one or more O-acyl isopeptide linkages is between a serine and the immediately preceding amino acid.

6. The peptide of claim 1, wherein the one or more O-acyl isopeptide linkages is between a threonine and the immediately preceding amino acid.

7. The peptide of claim 1, wherein the one or more O-acyl isopeptide linkages is between an allo-threonine and the immediately preceding amino acid.

8. The peptide of claim 1, wherein the one or more O-acyl isopeptide linkages is between a serine at residue position 7 and the immediately preceding amino acid.

9. The peptide of claim 1, wherein the one or more O-acyl isopeptide linkages is between a serine at residue position 3 and the immediately preceding amino acid.

10. The peptide of claim 1, wherein the one or more O-acyl isopeptide linkages is between a serine at residue position 7 and the immediately preceding amino acid and between a serine at residue position 3 and the immediately preceding amino acid.

11. The peptide of claim 1, wherein the peptide includes Lys, Arg, Leu, or allo-End at residue position 10.

12. The peptide of claim 1, wherein the peptide includes Thr or allo-Thr at residue position 7 or residue position 3, and wherein the one or more O-acyl isopeptide linkages is between the Thr or allo-Thr and the immediately preceding amino acid.

13. The peptide of claim 1, wherein the peptide includes a hydrophobic D-amino acid with an NH₂, N-methyl or N-alkyl substituent at residue position 1.

14. The peptide of claim 1, wherein the peptide includes a bulky hydrophobic amino acid at residue position 2, at residue position 6, or at residue position 11.

15. The peptide of claim 1, wherein the peptide includes a basic amino acid at residue position 4 or at residue position 9.

16. The peptide of claim 1, wherein the peptide includes a D-hydrophobic amino acid at residue position 5.

17. The peptide of claim 1, wherein the peptide includes aza-D-Thr at residue position 8.

18. The peptide of claim 1, wherein the peptide includes a macrolactam ring at residue positions 8 to 11.

19. The peptide of claim 1, wherein a fluorophore is attached to the peptide.

20. The peptide of claim 1, wherein the peptide sequence and structure are one of:

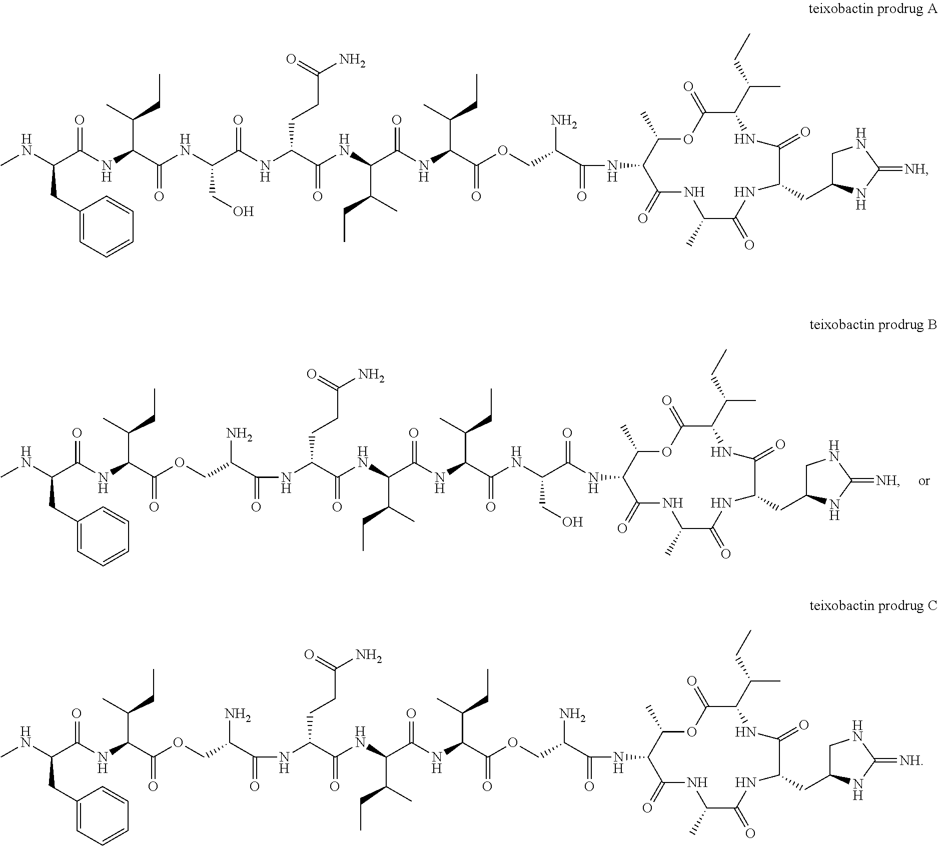

21. The peptide of claim 1, wherein the peptide sequence and structure are one of: 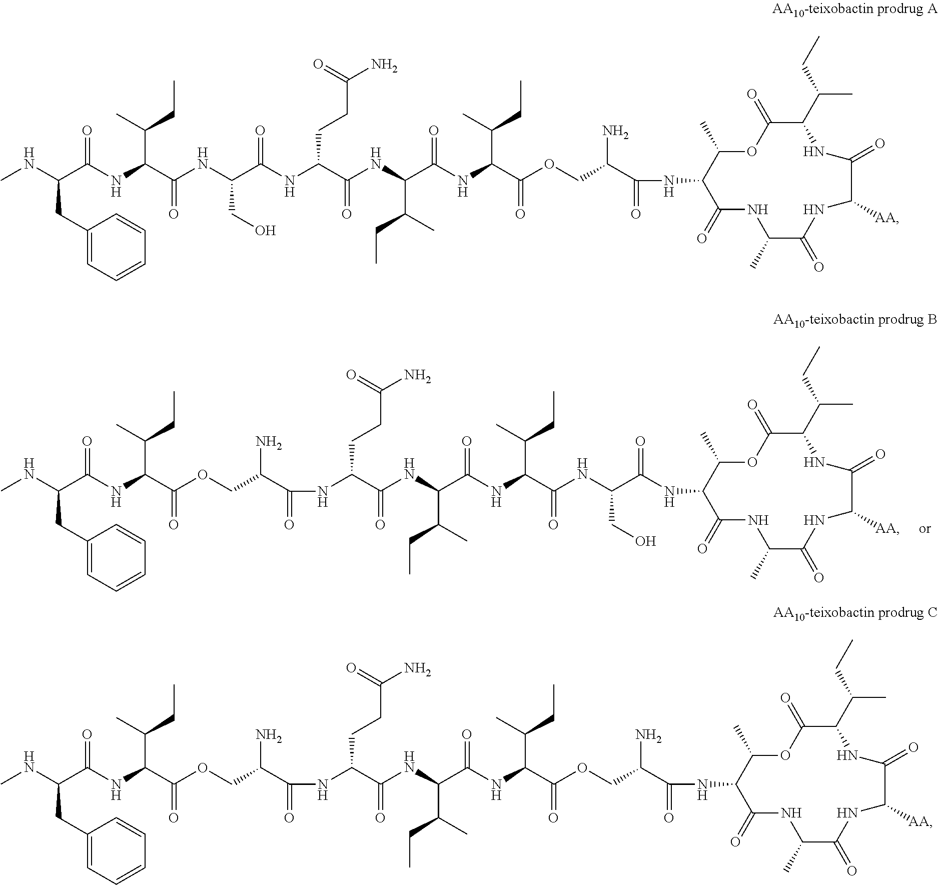
wherein AA is Lys, Arg, or Leu.

22. A medicament for the treatment of an infection of gram-positive bacteria, comprising:
a teixobactin peptide or a teixobactin analogue, or an enantiomer of the teixobactin peptide or the teixobactin analogue, having one or more O-acyl isopeptide linkages between two amino acids in place of an amide peptide bond.

23. The medicament of claim 22, wherein the one or more O-acyl isopeptide linkages is capable of chemically converting into amide peptide bonds at physiological conditions.

24. The medicament of claim 23, wherein chemical conversion of an O-acyl isopeptide linkage results in one of the two amino acids to have an alcohol side chain.

25. The medicament of claim 22, wherein the one or more O-acyl isopeptide linkages is between an amino acid with having a hydroxy group at the beta position and the immediately preceding amino acid.

26. The medicament of claim 22, wherein the one or more O-acyl isopeptide linkages is between a serine and the immediately preceding amino acid.

27. The medicament of claim 22, wherein the one or more O-acyl isopeptide linkages is between a threonine and the immediately preceding amino acid.

28. The medicament of claim 22, wherein the one or more O-acyl isopeptide linkages is between an allo-threonine and the immediately preceding amino acid.

29. The medicament of claim 22, wherein the one or more O-acyl isopeptide linkages is between a serine at residue position 7 and the immediately preceding amino acid.

30. The medicament of claim 22, wherein the one or more O-acyl isopeptide linkages is between a serine at residue position 3 and the immediately preceding amino acid.

31. The medicament of claim 22, wherein the one or more O-acyl isopeptide linkages is between a serine at residue position 7 and the immediately preceding amino acid and between a serine at residue position 3 and the immediately preceding amino acid.

32. The medicament of claim 22, wherein the peptide includes Lys, Arg, Leu, or allo-End at residue position 10.

33. The medicament of claim 22, wherein the peptide includes Thr or allo-Thr at residue position 7 or residue position 3, and wherein the one or more O-acyl isopeptide linkages is between the Thr or allo-Thr and the immediately preceding amino acid.

34. The medicament of claim 22, wherein the peptide includes a hydrophobic D-amino acid with an NH₂, N-methyl or N-alkyl substituent at residue position 1.

35. The medicament of claim 22, wherein the peptide includes a bulky hydrophobic amino acid at residue position 2, at residue position 6, or at residue position 11.

36. The medicament of claim 22, wherein the peptide includes a basic amino acid at residue position 4 or at residue position 9.

37. The medicament of claim 22, wherein the peptide includes a D-hydrophobic amino acid at residue position 5.

38. The medicament of claim 22, wherein the peptide includes aza-D-Thr at residue position 8.

39. The medicament of claim 22, wherein the peptide includes a macrolactam ring at residue positions 8 to 11.

40. The medicament of claim 22, wherein medicament is formulated for oral, topical, ocular, transdermal, transmucosal, parentenal, intranasal, pulmonary, epicutaneous, subcutaneous, intramuscular, or intravenous administration.

41. The medicament of claim 22, wherein the peptide sequence and structure are one of:

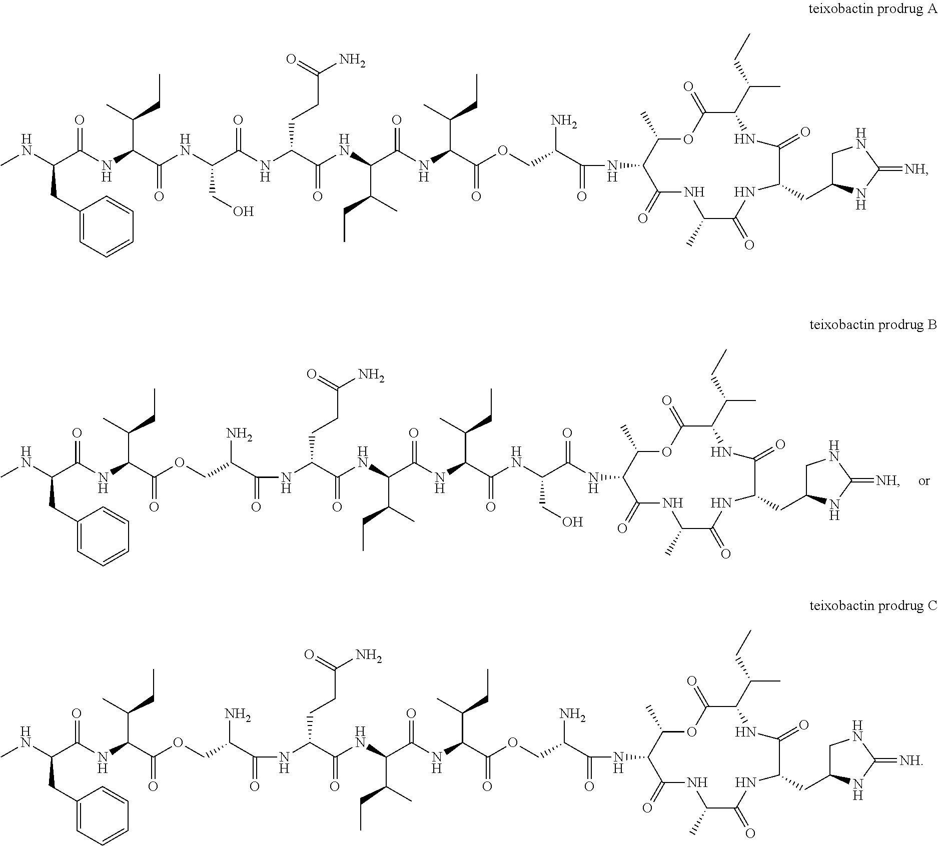

42. The medicament of claim 22, wherein the peptide sequence and structure are one of: 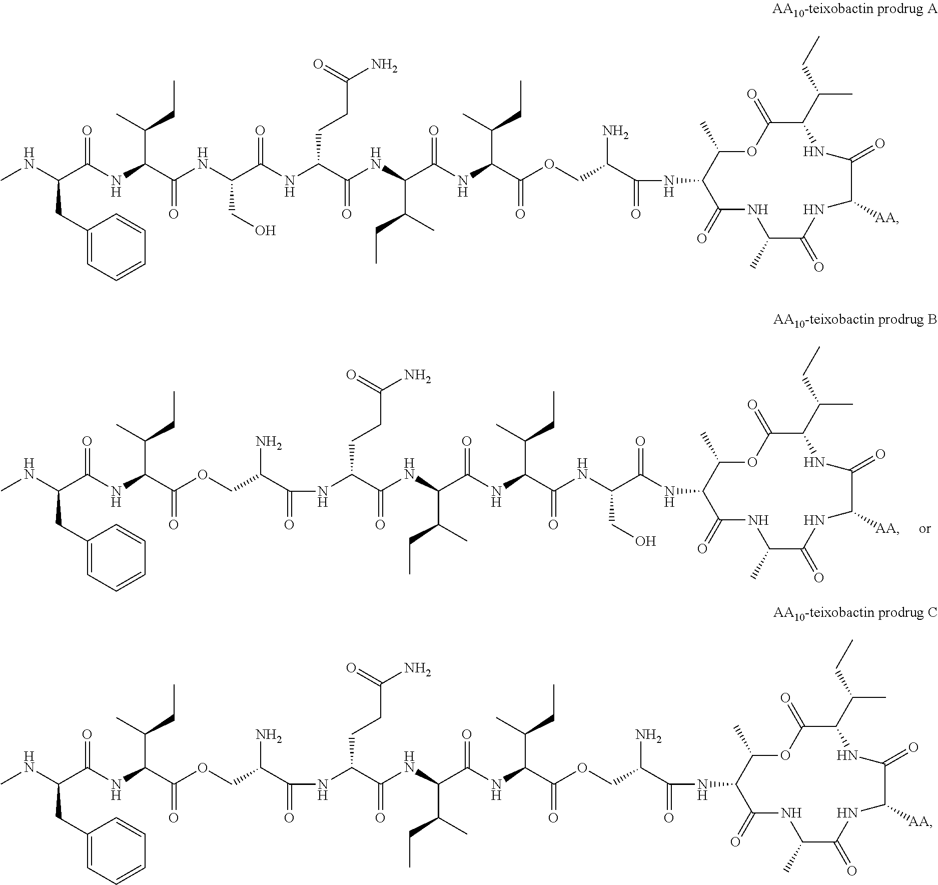
wherein AA is Lys, Arg, or Leu.

43. A method of treating an infection of gram-positive bacteria, comprising:
administering to a subject a teixobactin peptide or a teixobactin analogue, or an enantiomer of the teixobactin peptide or the teixobactin analogue, having one or more O-acyl isopeptide linkages between two amino acids in place of an amide peptide bond.

44. The method of claim 43, wherein the one or more O-acyl isopeptide linkages is capable of chemically converting into amide peptide bonds at physiological conditions.

45. The method of claim 44, wherein chemical conversion of an O-acyl isopeptide linkage results in one of the two amino acids to have an alcohol side chain.

46. The method of claim 43, wherein the one or more O-acyl isopeptide linkages is between an amino acid with having a hydroxy group at the beta position and the immediately preceding amino acid.

47. The method of claim 43, wherein the one or more O-acyl isopeptide linkages is between a serine and the immediately preceding amino acid.

48. The method of claim 43, wherein the one or more O-acyl isopeptide linkages is between a threonine and the immediately preceding amino acid.

49. The method of claim 43, wherein the one or more O-acyl isopeptide linkages is between an allo-threonine and the immediately preceding amino acid.

50. The method of claim 43, wherein the one or more O-acyl isopeptide linkages is between a serine at residue position 7 and the immediately preceding amino acid.

51. The method of claim 43, wherein the one or more O-acyl isopeptide linkages is between a serine at residue position 3 and the immediately preceding amino acid.

52. The method of claim 43, wherein the one or more O-acyl isopeptide linkages is between a serine at residue position 7 and the immediately preceding amino acid and between a serine at residue position 3 and the immediately preceding amino acid.

53. The method of claim 43, wherein the peptide incorporates Lys, Arg, Leu, or allo-End at residue position 10.

54. The method of claim 43, wherein the peptide incorporates Thr or allo-Thr at residue position 7 or residue position 3, and wherein the one or more O-acyl isopeptide linkages is between the Thr or allo-Thr and the immediately preceding amino acid.

55. The method of claim 43, wherein the peptide incorporates a hydrophobic D-amino acid with an NH₂, N-methyl or N-alkyl substituent at residue position 1.

56. The method of claim 43, wherein the peptide incorporates a bulky hydrophobic amino acid at residue position 2, at residue position 6, or at residue position 11.

57. The method of claim 43, wherein the peptide incorporates a basic amino acid at residue position 4 or at residue position 9.

58. The method of claim 43, wherein the peptide incorporates a D-hydrophobic amino acid at residue position 5.

59. The method of claim 43, wherein the peptide incorporates aza-D-Thr at residue position 8.

60. The method of claim 43, wherein the peptide incorporates a macrolactam ring at residue positions 8 to 11.

61. The method of claim 43, wherein the teixobactin peptide or the teixobactin analogue, or an enantiomer of the teixobactin peptide or the teixobactin analogue, is orally, topically, ocularly, transdermally, transmucosally, parentenally, intranasally, pulmonarily, epicutaneously, subcutaneously, intramuscularly, or intravenously administered.

62. The method of claim 43, wherein the peptide sequence and structure are one of:

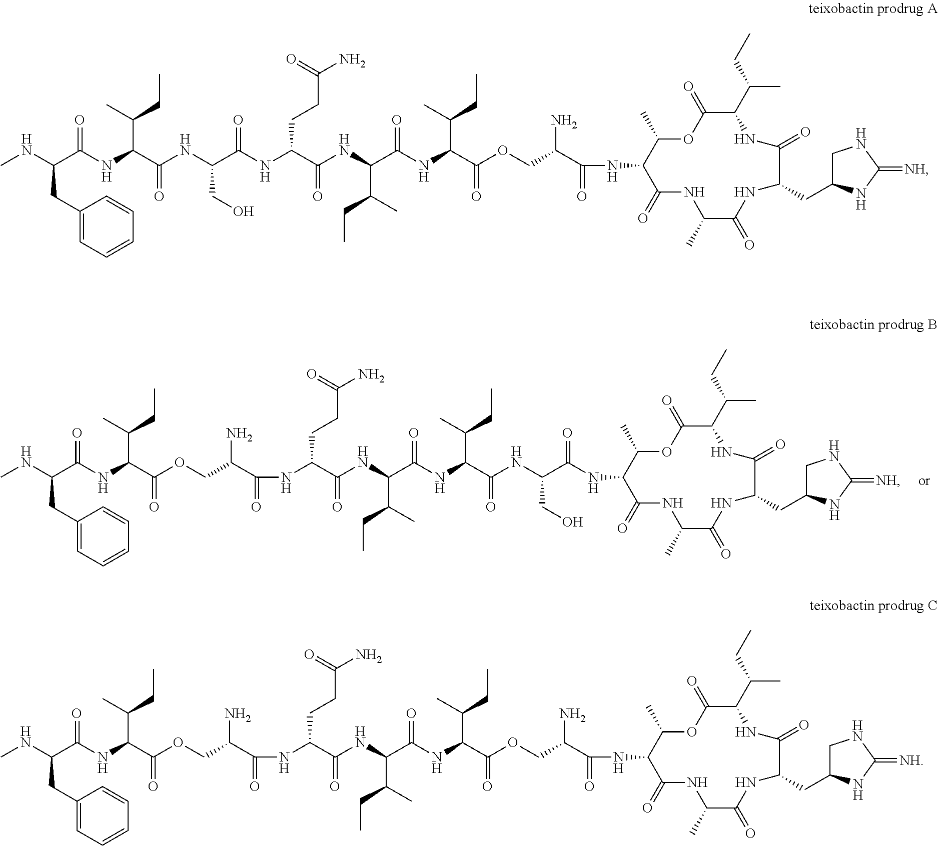

63. The method of claim 43, wherein the peptide sequence and structure are one of: 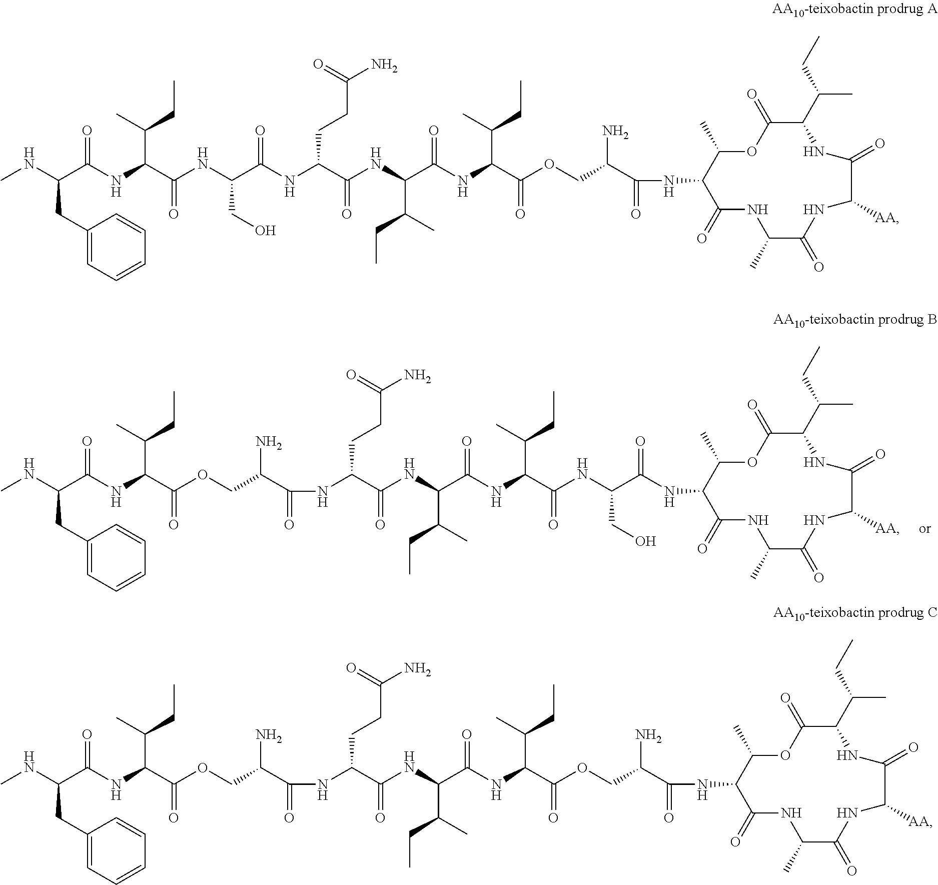
wherein AA is Lys, Arg, or Leu.

64. A teixobactin peptide or a teixobactin analogue, or an enantiomer of the teixobactin peptide or the teixobactin analogue, having one or more O-acyl isopeptide linkages between two amino acids in place of an amide peptide bond for treatment of an infection of gram-positive bacteria.

65. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the one or more O-acyl isopeptide linkages is capable of chemically converting into amide peptide bonds at physiological conditions.

66. The teixobactin peptide or a teixobactin analogue, or an enantiomer of the teixobactin peptide or the teixobactin analogue, having one or more O-acyl isopeptide linkages between two amino acids in place of an amide peptide bond of claim 65, wherein chemical conversion of an O-acyl isopeptide linkage results in one of the two amino acids to have an alcohol side chain.

67. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the one or more O-acyl isopeptide linkages is between an amino acid with having a hydroxy group at the beta position and the immediately preceding amino acid.

68. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the one or more O-acyl isopeptide linkages is between a serine and the immediately preceding amino acid.

69. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the one or more O-acyl isopeptide linkages is between a threonine and the immediately preceding amino acid.

70. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the one or more O-acyl isopeptide linkages is between an allo-threonine and the immediately preceding amino acid.

71. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the one or more O-acyl isopeptide linkages is between a serine at residue position 7 and the immediately preceding amino acid.

72. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the one or more O-acyl isopeptide linkages is between a serine at residue position 3 and the immediately preceding amino acid.

73. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the one or more O-acyl isopeptide linkages is between a serine at residue position 7 and the immediately preceding amino acid and between a serine at residue position 3 and the immediately preceding amino acid.

74. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the peptide incorporates Lys, Arg, Leu, or allo-End at residue position 10.

75. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the peptide incorporates Thr or allo-Thr at residue position 7 or residue position 3, and wherein the one or more O-acyl isopeptide linkages is between the Thr or allo-Thr and the immediately preceding amino acid.

76. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the peptide incorporates a hydrophobic D-amino acid with an NH₂, N-methyl or N-alkyl substituent at residue position 1.

77. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the peptide incorporates a bulky hydrophobic amino acid at residue position 2, at residue position 6, or at residue position 11.

78. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the peptide incorporates a basic amino acid at residue position 4 or at residue position 9.

79. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the peptide incorporates a D-hydrophobic amino acid at residue position 5.

80. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the peptide incorporates aza-D-Thr at residue position 8.

81. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the peptide incorporates a macrolactam ring at residue positions 8 to 11.

82. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the treatment comprises an administration that is oral, topical, ocular, transdermal, transmucosal, parentenal, intranasal, pulmonari, epicutaneous, subcutaneous, intramuscular, or intravenous.

83. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the peptide sequence and structure are one of:

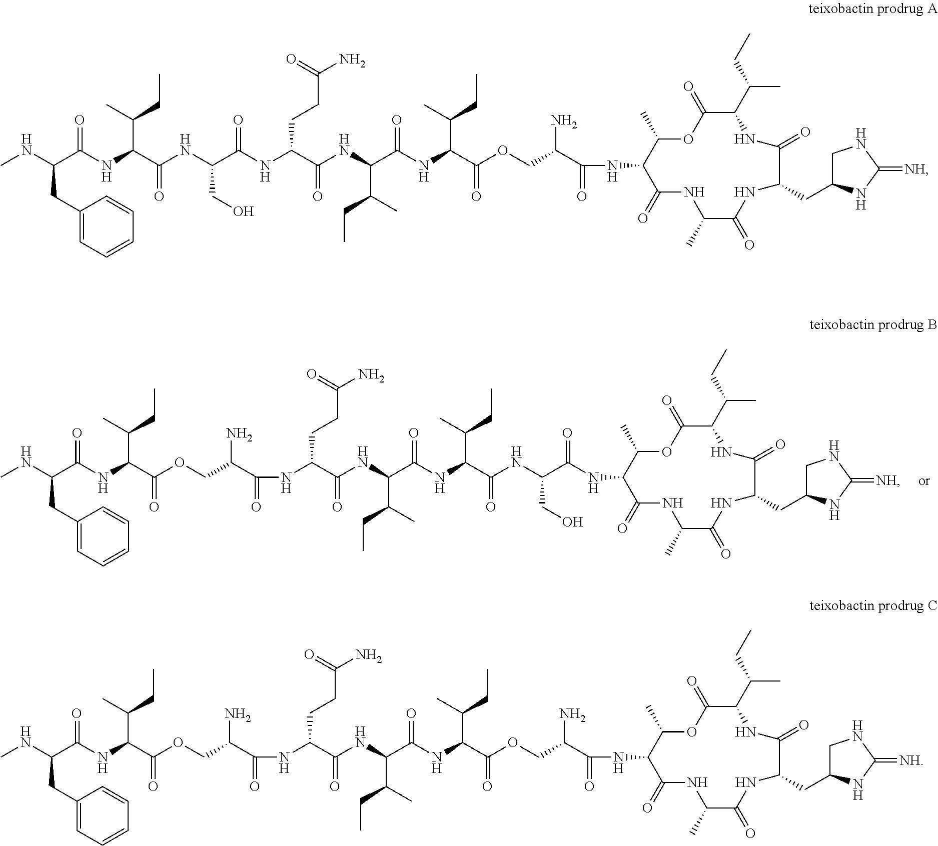

84. The teixobactin peptide or a teixobactin analogue, or an enantiomer of the teixobactin peptide or the teixobactin analogue, having one or more O-acyl isopeptide linkages between two amino acids in place of an amide peptide bond of claim 64, wherein the peptide sequence and structure are one of: 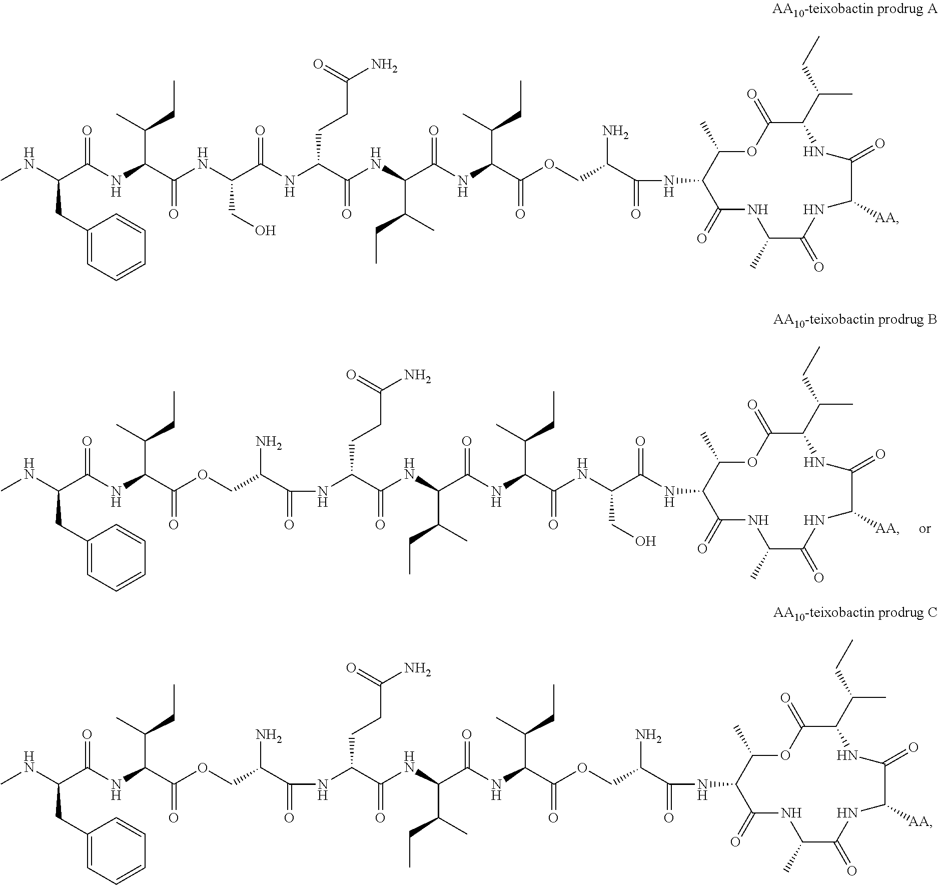
wherein AA is Lys, Arg, or Leu.


SEQUENCE LISTING (13 sequences)

SEQ ID NO: 1 - protein, 11 aa, Eleftheria terrae
  SITE 1: note = N-Me-D-Phe
  SITE 4: note = D-Gln
  SITE 5: note = D-allo-Ile
  SITE 8: note = D-Thr
  SITE 10: note = allo-enduracididine (allo-End)
FISQIISTAXI

SEQ ID NO: 2 - protein, 11 aa, synthetic construct
  REGION 1..11: note = modified AAs
  SITE 1: note = N-Me-D-Phe
  SITE 4: note = D-Gln
  SITE 5: note = D-allo-Ile
  SITE 7: note = O-acyl linked Ser
  SITE 8: note = D-Thr
  SITE 10: note = allo-enduracididine (allo-End)
FISQIISTAXI

SEQ ID NO: 3 - protein, 11 aa, synthetic construct
  REGION 1..11: note = modified AAs
  SITE 1: note = N-Me-D-Phe
  SITE 3: note = O-acyl linked Ser
  SITE 4: note = D-Gln
  SITE 5: note = D-allo-Ile
  SITE 8: note = D-Thr
  SITE 10: note = allo-enduracididine (allo-End)
FISQIISTAXI

SEQ ID NO: 4 - protein, 11 aa, synthetic construct
  REGION 1..11: note = modified AAs
  SITE 1: note = N-Me-D-Phe
  SITE 3: note = O-acyl linked Ser
  SITE 4: note = D-Gln
  SITE 5: note = D-allo-Ile
  SITE 7: note = O-acyl linked Ser
  SITE 8: note = D-Thr
  SITE 10: note = allo-enduracididine (allo-End)
FISQIISTAXI

SEQ ID NO: 5 - protein, 11 aa, synthetic construct
  REGION 1..11: note = modified AAs
  SITE 1: note = N-Me-D-Phe
  SITE 4: note = D-Gln
  SITE 5: note = D-allo-Ile
  SITE 7: note = O-acyl linked Ser
  SITE 8: note = D-Thr
FISQIISTAKI

SEQ ID NO: 6 - protein, 11 aa, synthetic construct
  REGION 1..11: note = modified AAs
  SITE 1: note = N-Me-D-Phe
  SITE 3: note = O-acyl linked Ser
  SITE 4: note = D-Gln
  SITE 5: note = D-allo-Ile
  SITE 8: note = D-Thr
FISQIISTAKI

SEQ ID NO: 7 - protein, 11 aa, synthetic construct
  REGION 1..11: note = modified AAs
  SITE 1: note = N-Me-D-Phe
  SITE 3: note = O-acyl linked Ser
  SITE 4: note = D-Gln
  SITE 5: note = D-allo-Ile
  SITE 7: note = O-acyl linked Ser
  SITE 8: note = D-Thr
FISQIISTAKI

SEQ ID NO: 8 - protein, 11 aa, synthetic construct
  REGION 1..11: note = modified AAs
  SITE 1: note = N-Me-D-Phe
  SITE 4: note = D-Gln
  SITE 5: note = D-allo-Ile
  SITE 7: note = O-acyl linked Ser
  SITE 8: note = D-Thr
FISQIISTARI

SEQ ID NO: 9 - protein, 11 aa, synthetic construct
  REGION 1..11: note = modified AAs
  SITE 1: note = N-Me-D-Phe
  SITE 3: note = O-acyl linked Ser
  SITE 4: note = D-Gln
  SITE 5: note = D-allo-Ile
  SITE 8: note = D-Thr
FISQIISTARI

SEQ ID NO: 10 - protein, 11 aa, synthetic construct
  REGION 1..11: note = modified AAs
  SITE 1: note = N-Me-D-Phe
  SITE 3: note = O-acyl linked Ser
  SITE 4: note = D-Gln
  SITE 5: note = D-allo-Ile
  SITE 7: note = O-acyl linked Ser
  SITE 8: note = D-Thr
FISQIISTARI

SEQ ID NO: 11 - protein, 11 aa, synthetic construct
  SITE 1: note = N-Me-D-Phe
  SITE 4: note = D-Gln
  SITE 5: note = D-allo-Ile
  SITE 7: note = O-acyl linked Ser
  SITE 8: note = D-Thr
FISQIISTALI

SEQ ID NO: 12 - protein, 11 aa, synthetic construct
  SITE 1: note = N-Me-D-Phe
  SITE 3: note = O-acyl linked Ser
  SITE 4: note = D-Gln
  SITE 5: note = D-allo-Ile
  SITE 8: note = D-Thr
FISQIISTALI

SEQ ID NO: 13 - protein, 11 aa, synthetic construct
  SITE 3: note = O-acyl linked Ser
  SITE 4: note = D-Gln
  SITE 5: note = D-allo-Ile
  SITE 7: note = O-acyl linked Ser
  SITE 8: note = D-Thr
  SITE 1: note = N-Me-D-Phe
FISQIISTALI